(19) United States
(12) Patent Application Publication
(10) Pub. No.: US 2010/0267062 A1
(43) Pub. Date: Oct. 21, 2010

(54) Osteopontin as Novel Prognostic Biomarker for Heart Failure

(76) Inventors: Norbert Frey, Kronshagen (DE); Mark Rosenberg, Kiel (DE); Hugo Katus, Heidelberg (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/678,216

(22) PCT Filed: Sep. 26, 2008
(86) PCT No.: PCT/EP08/08232

§ 371 Date: Jul. 6, 2010

Related U.S. Application Data
(60) Provisional application No. 60/975,181, filed on Sep. 26, 2007.

(51) Int. Cl. G01N 33/53; C12Q 1/02; G01N 33/48; G01N 33/58

(52) U.S. Cl. 435/7.92; 435/29; 436/87; 435/7.1

(57) ABSTRACT

The present invention relates to methods for providing a diagnosis, prognosis and/or risk stratification of a subject with heart failure, comprising determining the concentration of osteopontin (OPN) in the biological sample, preferably in a plasma sample. An OPN cut-off value is discloses as a valuable reference value. The present invention furthermore relates to the use of osteopontin as marker for diagnosis, prognosis and/or risk stratification of a subject with heart failure, the use of the determination of the osteopontin plasma concentration in a biological sample of a subject for diagnosis, prognosis and/or risk stratification of heart failure as well as kits for performing the methods and uses of the invention. The present invention allows particularly for risk stratification of patients with heart failure, such as mortality prediction and prognosis of heart failure severity.

The present invention relates to methods for providing a diagnosis, prognosis and/or risk stratification of a subject with heart failure, comprising determining the concentration of osteopontin (OPN) in the biological sample, preferably in a plasma sample. An OPN cut-off value is discloses as a valuable reference value. The present invention furthermore relates to the use of osteopontin as marker for diagnosis, prognosis and/or risk stratification of a subject with heart failure, the use of the determination of the osteopontin plasma concentration in a biological sample of a subject for diagnosis, prognosis and/or risk stratification of heart failure as well as kits for performing the methods and uses of the invention. The present invention allows particularly for risk stratification of patients with heart failure, such as mortality prediction and prognosis of heart failure severity.

### BACKGROUND OF THE INVENTION

Heart failure is a highly prevalent syndrome throughout the industrialized world, associated with significant morbidity and mortality. In the United States, heart failure affects more than five million people and is responsible for nearly 50.000 deaths each year (1). Furthermore, annual hospitalizations for heart failure have increased over the last 20 years from 377.000 to almost one million (2).

Thus, in patients with heart failure, an accurate diagnosis and prognostic evaluation is critical in order to identify those at greatest risk for cardiac decompensation and death. Traditional risk stratification by clinical parameters as well as assessment of left ventricular ejection fraction has been proven to be helpful in the clinical management of heart failure patients (3). More recently, the natriuretic peptides, in particular brain natriuretic peptide (BNP) or its fragment N-terminal prohormone BNP (NT-pro-BNP), have emerged as biomarkers that convey additional information for diagnosis and prognostication of mortality (4). However, even when clinical information is combined with BNP levels, there is considerable variation in the outcome (5). As a consequence, there still is a great interest for new biomarkers that complement existing diagnostic tools and may facilitate risk stratification in patients with heart failure.

Osteopontin (OPN) is a 32 kDa glycoprotein expressed in various cell types, including cardiomyocytes and fibroblasts. OPN can exist as an immobilized extracellular matrix molecule or as soluble cytokine and contains an RGD (arginin-glycin-aspartate) binding sequence that mediates interaction with several integrins, including β1-integrin, which is predominantly expressed in the myocardium (6) (see FIG. 1).

Because of its localization and molecular properties, osteopontin has been suggested to be involved in the communication between the extracellular matrix and cardiomyocytes (reviewed in (7)). Moreover, the inventors and others have shown that OPN is upregulated in several animal models of cardiac hypertrophy and failure (8-9) (see FIG. 2), implying a role in myocardial remodelling in response to biomechanical stress.

Stawowy et al. (10) analyzed the expression of osteopontin in myocardial biopsies obtained from a small patient group of 10 patients with dilated cardiomyopathy (DCM). They found a significant upregulation of osteopontin in cardiac myocytes compared to control tissue. Furthermore, myocardial osteopontin content correlated positively with left ventricular endsystolic/enddiastolic volume index (LVESVI, LVEDVI), left ventricular enddiastolic pressure (LVEDP) and myocyte diameter (MD). Negative correlations were found for myocardial osteopontin and left or right ventricular ejection fraction (LVEF, RVEF).

Satoh et al. (11) describe that osteopontin mRNA levels were elevated in myocardium obtained from 51 patients with dilated cardiomyopathy and positively correlated to collagen I mRNA. Furthermore, it was found that mRNA levels of osteopontin in cardiac myocytes and mRNA levels of collagen I are related to left ventricular dimensions and systolic functions of patients suffering from a dilated cardiomyopathy.

Suezawa et al. (12) evaluated the relationship of osteopontin plasma levels in the course of an acute myocardial infarction. In this study osteopontin plasma levels were found to be elevated and correlated with left ventricular dysfunction and volume. A small group of 18 patients with myocardial infarction who underwent successful reperfusion after anterior-wall acute myocardial infarction were the study objects.

The present invention aims to improve the diagnosis present in the prior art and it is, thus, an object of the present invention to provide improved methods and means which allow for diagnosis and furthermore allow for prognosis and/or risk stratification of patients with heart failure independent on the etiology of the heart failure.

### SUMMARY OF THE INVENTION

According to the present invention this object is solved by a method for providing a diagnosis, prognosis and/or risk stratification of a subject with heart failure, comprising determining the concentration of osteopontin (OPN) in the biological sample, preferably in a plasma sample.

The inventive method for providing a diagnosis, prognosis and/or risk stratification of a subject with heart failure preferably comprises the following steps:
a) providing a biological sample from the subject;
b) determining the concentration of osteopontin (OPN) in said sample,
c) comparing the determined OPN concentration with at least one reference value, and
d) optional, assessing at least one further biomarker for heart failure.

The object is further solved by the use of the methods according to the invention for identifying of patients and patient subgroups with elevated OPN concentrations, which suffer from a significantly higher cardiac risk.

The object is further solved by the use of osteopontin as marker for a diagnosis, prognosis and/or risk stratification of a subject with heart failure.

The object is further solved by the use of the determination of the osteopontin plasma concentration in a biological sample of a subject for diagnosis, prognosis and/or risk stratification of heart failure.

The object is further solved by a kit for performing the methods and uses according to the invention.

The present invention and its preferred embodiments are described in more detail below.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

As outlined above, the present invention provides a method for providing a diagnosis, prognosis and/or risk stratification of a subject with heart failure.

The methods of the present invention are characterized in that osteopontin concentration is determined and used as a novel biomarker for diagnosis, prognosis and/or risk stratification of heart failure.

“Heart failure” or “congestive heart failure” (CHF) is a condition that can result from any structural or functional cardiac disorder that impairs the ability of the heart to fill with or pump a sufficient amount of blood throughout the body. Causes and contributing factors to congestive heart failure include the following (with specific reference to left (L) or right (R) sides): genetic family history of CHF, ischemic heart disease/myocardial infarction (coronary artery disease), thyrotoxicosis (hyperthyroidism), hypothyroidism, anemia, arrhythmia, hypertension, infection, cardiac fibrosis, coarctation of the aorta (L), aortic stenosis/regurgitation (L), mitral regurgitation (L), pulmonary stenosis/pulmonary hypertension/cor pulmonale/pulmonary embolism (R), mitral valve disease (L), cardiomyopathy, including noncompaction cardiomyopathy (L&R).

According to the present invention, “heart failure” is selected from the group of chronic heart failure, such as chronic heart failure due to dilated or ischemic cardiomyopathy; systolic heart failure; dilated cardiomyopathy (DCM); ischemic cardiomyopathy; acute myocardial infarction; left ventricular dysfunction and right ventricular dysfunction.

Cardiomyopathy, which literally means “heart muscle disease”, is the deterioration of the function of the myocardium for any reason. Cardiomyopathies can generally be categorized into two groups, based on WHO guidelines: extrinsic and intrinsic cardiomyopathies.

Extrinsic cardiomyopathies are cardiomyopathies where the primary pathology is outside the myocardium itself. Most cardiomyopathies are extrinsic, because by far the most common cause of a cardiomyopathy is ischemia. Ischemic cardiomyopathy, for instance, is a weakness in the muscle of the heart due to inadequate oxygen delivery to the myocardium with coronary artery disease being the most common cause.

An intrinsic cardiomyopathy is weakness in the muscle of the heart that is not due to an identifiable external cause. The term intrinsic cardiomyopathy does not describe the specific etiology of weakened heart muscle. The intrinsic cardiomyopathies are a heterogeneous group of disease states, each with their own causes. Intrinsic cardiomyopathy has a number of causes including drug and alcohol toxicity, certain infections (including hepatitis C), and various genetic and idiopathic (i.e., unknown) causes. There are four main types of intrinsic cardiomyopathy: first, dilated cardiomyopathy (DCM), the most common form, and one of the leading indications for heart transplantation. In DCM the heart (especially the left ventricle) is enlarged and the pumping function is diminished. Second, hypertrophic cardiomyopathy (HCM or HOCM), a genetic disorder caused by various mutations in genes encoding sarcomeric proteins. In HCM the heart muscle is thickened, which can obstruct blood flow and prevent the heart from functioning properly. Third, arrhythmogenic right ventricular cardiomyopathy (ARVC) arises from an electrical disturbance of the heart in which heart muscle is replaced by fibrous scar tissue. The right ventricle is generally most affected. Fourth, restrictive cardiomyopathy (RCM) is the least common cardiomyopathy. The walls of the ventricles are stiff, but may not be thickened, and resist the normal filling of the heart with blood. Furthermore, noncompaction cardiomyopathy a more recent form of cardiomyopathy is recognized as its own separate type since the 1980's. It refers to a cardiomyopathy where the left ventricle wall has failed to properly grow from birth and such has a spongy appearance when viewed during an echocardiogram.

Cardiomyopathies with secondary cause are also comprised, such as cardiac amyloidosis.

Also heart failure conditions due to pulmonary arterial hypertension (PAH) are comprised.

As used herein, providing a diagnosis of a subject is determining heart failure, namely independent on the etiology of the heart failure, i.e. determining whether or not a subject has suffered heart failure recently or in the past.

Due to the findings of the inventors that osteopontin levels, i.e. the osteopontin plasma concentrations, are significantly increased in patients with heart failure, in fact irrespective of the underlying etiology of the heart failure, osteopontin is a diagnosis marker for heart failure. Although not being specific for heart muscle, the OPN concentration in a biological sample, preferably the osteopontin plasma concentration, can be used for diagnosing heart failure. See below for further details.

As used herein, providing a prognosis of a subject is preferably selected from determining heart failure severity, risk for subsequent all-cause mortality and risk assessment of the subject with heart failure.

Due to the findings of the inventors that osteopontin levels, i.e. the osteopontin plasma concentrations, correlate with the severity of heart failure and that the osteopontin level, i.e. the osteopontin plasma concentration, predicts mortality in patients with heart failure, osteopontin is a prognosis marker for heart failure and the OPN concentration in a biological sample, preferably the osteopontin plasma concentration, can be used for the prognosis of heart failure. See below for further details.

Preferably, the prognosis with respect to the risk for subsequent all-cause mortality is the 4-year mortality prediction.

Throughout the specification, a prediction or estimation of subsequent all-cause mortality can also be called a prediction or estimation of the survival rates of patients.

“Risk assessment” or “risk stratification” of subjects with heart failure according to the present invention refers to the evaluation of factors, such as biomarkers, in order to predict the risk of future events or even death and in order to decide about the type, manner, dosis, regimen of therapy and treatment for the individual subject.

In a step a) of a preferred embodiment of the method of the present invention a biological sample from the subject is obtained or provided.

A “biological sample” according to the present invention is preferably taken from a mammal, more preferably a human.

A “subject” according to the present invention is preferably a mammal, more preferably a human.

The biological samples within the meaning of the present invention are samples of a subject with heart failure as well as control samples, as described in further detail below.

The biological sample is preferably selected from peripheral blood or fractions thereof and cell culture suspensions or fractions thereof.

The biological sample is preferably selected from a bodily fluid, whole blood, plasma, serum or urine.

Preferably, the sample is blood plasma or blood serum, more preferably plasma.

In an embodiment of the invention the biological sample was pre-treated, for instance a coagulation inhibitor, such as heparin or EDTA, was added.

Methods for obtaining and/or providing the above biological samples are known in the art.

In a subsequent step b) of a preferred embodiment of the method of the present invention the concentration of osteopontin (OPN) in said sample is determined.

Osteopontin (OPN) according to the present invention refers to a 32 kDa glycoprotein with mammalian origin, preferably human OPN. OPN is expressed in various cell types, including cardiomyocytes, osteoblasts, vascular muscle cells and fibroblasts. OPN can be present in the extracellular matrix as well as in a soluble form. OPN contains an RGD (arginine-glycin-aspartate) binding sequence that mediates interaction with several surface receptors, e.g. integrins, including β1-integrin, which is predominantly expressed in the myocardium (6) (see FIG. 1).

For a preferred nucleotide sequence and amino acid sequence of human OPN see SEQ ID NO: 1 and SEQ ID NO: 2, respectively. The Genbank accession number is NM_{—}001040060.

Furthermore, two splice variants of human osteopontin have been described which differ from one another by the presence or absence of 14 amino acids after position 58 in the pre-signal-processed protein. CC1074 is the fully active mature chain (aa 17-314) which contains the full sized splice variant at aa 59-72 (see Protein accession number S09575; see (13)).

Preferably, in the methods and uses of the present invention the concentration of human OPN in the soluble form is determined.

More preferably, in the methods and uses of the present invention the plasma concentration of human OPN in the soluble form is determined, i.e. the concentration of the soluble form of OPN in plasma samples.

In a subsequent step c) the determined OPN concentration, i.e. the measured osteopontin concentration, is compared with at least one reference value.

### Reference Value

“Reference value” is a term used in medicine to denote a laboratory value used as a reference for values/data obtained by laboratory examinations of patients or samples collected from patients.

According to the present invention the reference value is the OPN concentration of a control sample or a osteopontin cut-off value.

### a) (Median) OPN Concentration of a Control Sample as Reference Value

In a preferred embodiment of the present invention the reference value is the osteopontin concentration of a control sample.

The reference value is preferably the OPN plasma concentration of a control sample.

The control sample is preferably selected from the biological sample of a control subject, or biological samples of a group of control subjects.

The biological sample(s) is(are) preferably a plasma sample(s).

A “control subject” (which can also be called a “healthy subject”) according to the present invention is a subject, e.g. a patient, without signs of a significant heart disease or heart failure.

Preferably, subjects, e.g. patients, are determined to be “control” subjects according to the present invention after they undergo coronary angiography for suspected coronary artery disease (CAD). Subjects are preferably only considered to be “healthy” subjects if invasive examination excludes CAD as well as systolic or diastolic dysfunction (defined as left ventricular enddiastolic pressures of less than 12 mmHg). Subjects who fulfill these criteria can be preferably still be excluded from “control” subjects, if valvular heart disease or myocardial hypertrophy are evident on echocardiography. Further requirements are preferably the absence of other acute or chronic diseases, as well as normal results on routine laboratory testing.

The OPN concentration of a control sample is preferably the median OPN concentration of control samples of a group of control subjects, i.e. the mean value of the OPN concentrations of control samples of a group of control subjects.

A median OPN concentration is preferably obtained from a group of at least 20 control subjects, more preferably at least 30, even more preferably at least 40.

The median OPN concentration is preferably the median OPN plasma concentration of a control sample.

OPN Concentration is Elevated in Subjects with Heart Failure

The OPN concentration of a subject with heart failure is elevated compared to the reference value, i.e. the OPN concentration of a control sample as defined herein.

Preferably, the osteopontin plasma concentration of a subject with heart failure is elevated compared to the reference value, i.e. the osteopontin plasma concentration of a control sample as defined herein.

A preferred osteopontin plasma concentration of a control sample as reference value is higher than 300 ng/ml plasma, preferably higher than 350 ng/ml plasma and more preferably 382 ng/ml plasma, wherein the 25^{th}-75^{th} percentile range is 257 ng/ml to 540 ng/ml.

The OPN concentration of a subject with heart failure is elevated compared to the reference value when it is significantly higher.

The osteopontin plasma concentration of a subject with heart failure is elevated compared to the reference value when it is significantly higher. Preferably, the OPN plasma concentration of a subject with heart failure is at least 20% higher (1.2 fold), more preferably at least 30% (1.3 fold) higher, even more preferably at least 40% higher (1.4 fold). The OPN plasma concentration of a subject with heart failure can also be 100% higher (2.0 fold) or more.

In a preferred embodiment (see also Examples): the median osteopontin plasma level in the control sample was 382 ng/ml (25^{th}-75^{th} percentile range: 257 ng/ml-540 ng/ml). Patients with systolic heart failure displayed a significantly higher (p<0.01) median osteopontin plasma level of 532 ng/ml (232 ng/ml-875 ng/ml). The upregulation of osteopontin plasma levels was independent of the underlying heart failure etiology. Patients with dilated cardiomyopathy revealed a median osteopontin plasma level of 577 ng/ml (151 ng/ml-954 ng/ml) that did not significantly differ from the median level of patients with ischemic cardiomyopathy (508 ng/ml (310 ng/ml-791 ng/ml)). (see also Table 1). Neither in heart failure patients nor in controls a significant difference between male and female subjects was observed (data not shown).

In another preferred embodiment (see also Example 3):

OPN was significantly elevated in patients with pulmonary arterial hypertension compared to healthy controls (720 ng/ml vs. 382 ng/ml; p<0.0001). Furthermore, OPN levels were higher in patients with moderate to severe heart failure compared to patients with no or mild symptoms (WHO Fc III/IV 866 ng/ml vs. WHO Fc I/II 686 ng/ml; p=0.03). Patients with a right ventricular enddiastolic diameter above 30 mm displayed higher OPN plasma levels (789 ng/ml vs. 512 ng/ml; p<0.001). Additionally, plasma levels of OPN above the median (766 ng/ml) reliably predicted right ventricular dysfunction in our patient cohort (OR 6.0; 95%-CI 2.0-18.4; p=0.001). In a multivariate analysis including demographical, clinical and biochemical parameters such as NT-pro-BNP, OPN emerged also as an independent predictor of right ventricular dysfunction (OR 5.3; 95%-CI 1.1-27.1; p=0.04).

### b) Osteopontin Cut-Off Value as Reference Value

In another preferred embodiment the reference value is an osteopontin cut-off value.

Preferably the osteopontin cut-off value was determined by a statistical classification method, preferably receiver operating curve (ROC) analysis, from biological samples of a patient group.

The biological samples are preferably plasma samples.

Receiver Operating Curve (ROC) Analysis

ROC is known in the art of medicine.

Briefly, the ability of a test to discriminate diseased cases from normal cases is evaluated using Receiver Operating Characteristic (ROC) curve analysis. ROC curves can also be used to compare the diagnostic performance of two or more laboratory or diagnostic tests. When the results of a particular test in two populations is considered, one population with a disease, the other population without the disease, a perfect separation between the two groups is rarely observed. Indeed, the distribution of the test results will overlap.

For every possible cut-off point or criterion value selected to discriminate between the two populations, there will be some cases with the disease correctly classified as positive (TP=True Positive fraction), but some cases with the disease will be classified negative (FN=False Negative fraction). On the other hand, some cases without the disease will be correctly classified as negative (TN=True Negative fraction), but some cases without the disease will be classified as positive (FP=False Positive fraction).

The schematic outcomes of a test are:

| | Disease | | Disease | | |
|---|---|---|---|---|---|
| Test | Present | n | Absent | n | Total |
| Positive | True Positive | a | False | c | a + c |
| | | | Positive | | |
| Negative | False | b | True | d | b + d |
| | Negative | | Negative | | |
| Total | | a + b | | c + d | |

Sensitivity: probability that a test result will be positive when the disease is present (true positive rate, expressed as a percentage). (=a/(a+b))

Specificity: probability that a test result will be negative when the disease is not present (true negative rate, expressed as a percentage). (=d/(c+d))

Positive likelihood ratio: ratio between the probability of a positive test result given the presence of the disease and the probability of a positive test result given the absence of the disease. (=sensitivity/(1−specificity)

Negative likelihood ratio: ratio between the probability of a negative test result given the presence of the disease and the probability of a negative test result given the absence of the disease. (=specificity/(1−sensitivity)

Positive predictive value: probability that the disease is present when the test is positive (expressed as a percentage). (=a/(a+c))

Negative predictive value: probability that the disease is not present when the test is negative (expressed as a percentage). (=d/(b+d))

Thus, as used herein “ROC analysis” relates to a statistical method to quantify how accurately a diagnostic test performs when it is required to make a series of discriminations into two different states (diseased and non-diseased) on the basis of a certain diagnostic parameter. Every value of that discriminating parameter is used as a cut-off with calculation of the corresponding sensitivity and specificity.

A cut-off value determined by ROC analysis is an “optimized” value.

Patient Group

The patient group used to obtain an osteopontin cut-off value comprises subjects with heart failure and healthy subjects (control subjects). Thus, the patient group comprises a first subgroup of subjects with heart failure and a second subgroup of healthy subjects (control subjects).

The first subgroup of subjects with heart failure comprises subjects with heart failure of different etiology, such as dilated cardiomyopathy and ischemic cardiomyopathy.

The second subgroup comprises healthy subjects (control subjects) as defined above, i.e. subjects that show no signs of heart failure.

Preferably both subgroups have the same age distribution.

Preferably there are exclusion criteria for the subgroups, such as malignant diseases, inflammatory diseases or renal failure.

Preferably there are inclusion criteria for the first subgroup (subjects with heart failure): minimum age, such as 18 years; medication, such as ACE inhibitor or angiotensin II receptor blocker; significantly reduced left ventricular systolic function, such as with an ejection fraction of less than 40%.

In a preferred embodiment (see also Examples), heart failure patients (subgroup 1) can be recruited from the heart failure clinic of a large university hospital. Eligible patients are ≧18 years of age and reveal significantly reduced left ventricular systolic function with an ejection fraction of less than 40%. Patients with dilated cardiomyopathy or ischemic heart failure are both included. As angiotensin II extensively stimulates osteopontin expression in the heart, all patients have to be on an ACE inhibitor or angiotensin II receptor blocker. Patients with malignant or inflammatory diseases, history of organ transplantation and significant acute/chronic renal failure (serum creatinine>2 mg/dl) are excluded. According to the inclusion and exclusion criteria a database inquiry of the heart failure clinic can be performed. Plasma samples can be considered for osteopontin testing, if they were drawn within the past 10 years (1996-2006).

In a preferred embodiment the osteopontin cut-off value is higher than 850 ng/ml plasma, preferably higher than 900 ng/ml plasma, more preferably the osteopontin cut-off value is 929 ng/ml plasma.

The preferred OPN cut-off value is 929 ng/ml with a sensitivity of 46% and a specificity of 83%. This preferred OPN cut-off value was determined by ROC analysis.

A cut-off value determined by ROC analysis is an “optimized” value.

The preferred osteopontin cut-off value was obtained from a patient group that consisted of 420 heart failure patients and 43 healthy controls. In order to assay osteopontin plasma levels in patients with significant heart failure, 420 patients of whom 267 had dilated cardiomyopathy (64%) and 153 an ischemic origin of heart failure (36%) were analyzed (first subgroup). The median age of the heart failure group was 57 years and included 342 men (81%) and 78 women (19%). The control group (second subgroup) was comprised of 17 men (39.5%) and 26 women (60.5%) with a median age of 59 years (see FIG. 3).

A preferred cut-off value of OPN for the prediction of all cause mortality within 48 months in that patient group is 929 ng/ml with a sensitivity of 46% and a specificity of 83% (see Examples and FIG. 4).

The OPN cut-off value is preferably used for estimating the survival rates of patients. In the above patient or study group:

Estimated survival rates after 12, 24, 36 and 48 months in heart failure patients with osteopontin levels above the cut-off value of 929 ng/ml were 68%, 58%, 48%, 43% vs. 90%, 81%, 77%, 71% in the population with lower osteopontin levels. As a result there was a significant difference in estimated 4 year mortality rates between the groups (Hazard Ratio (HR) 3.4; 95%-CI 2.2-5.3; p<0.0001). Median survival of patients measured with high osteopontin values was only 34 months, whereas median survival in patients with lower osteopontin levels was more than 48 months. See also Table 1 below.

TABLE 1

| | OPN conc. | OPN conc. |
|---|---|---|
| Estimated survival rates | <cut-off value | >cut-off value |
| After 12 months | 90% | 68% |
| After 24 months | 81% | 58% |
| After 36 months | 77% | 48% |
| After 48 months | 71% | 43% |

Thus, a OPN concentration below the cut-off value of 929 ng/ml plasma is predictive of a survival of:
about 90% of patients after 12 months;
about 80% of patients after 24 months;
more than 70%, more preferably about 75% of patients after 36 months; and
about 70% of patients after 48 months.

Whereas, a OPN concentration above the cut-off value of 929 ng/ml plasma is predictive of a survival of
more than 60%, more preferably about 65% of patients after 12 months;
more than 50%, more preferably about 55% of patients after 24 months;
more than 40%, more preferably about 45% of patients after 36 months; and
about 40% of patients after 48 months.

In a preferred embodiment the OPN concentration of the subject is elevated compared to the reference value, i.e. the osteopontin cut-off value as defined herein.

Preferably, the osteopontin plasma concentration of a subject with heart failure is elevated compared to the reference value, i.e. the osteopontin cut-off value as defined herein.

The OPN concentration of a subject with heart failure is elevated compared to the reference value when it is significantly higher. The osteopontin plasma concentration of a subject with heart failure is elevated compared to the reference value when it is significantly higher.

In an optional step d) at least one further biomarker for heart failure is assessed.

### Assessment of Further Biomarkers for Heart Failure

In a preferred embodiment the method of the present invention further comprises the assessment of at least one further biomarker for heart failure.

Several biomarkers for heart failure are known in the art, such as NYHA stage, 6 minute walk test, maximum oxygen uptake assessed during ergospirometry (VO₂ max), age, brain natiuretic peptide (BNP), NT-pro-BNP, soluble CD40 ligand (sCD40L), PAPP-A, troponin T (TnT), MPO, VEGF and P1GF and creatinine, in particular serum creatinine.

A preferred at least one further biomarker for heart failure is NYHA stage, age, brain natiuretic peptide (BNP) and NT-pro-BNP and creatinine, in particular serum creatinine.

NYHA

The New York Heart Association (NYHA) Functional Classification provides a simple way of classifying the extent of heart failure. It places patients in one of four categories based on how much they are limited during physical activity:
Stage I No symptoms and no limitation in ordinary physical activity.
Stage II Mild symptoms and slight limitation during ordinary activity. Comfortable at rest.
Stage III Marked limitation in activity due to symptoms, even during less-than-ordinary activity. Comfortable only at rest.
Stage IV Severe limitations. Experiences symptoms even while at rest.

BNP/NT-pro-BNP

Brain natriuretic peptide (BNP, also known as B-type natriuretic peptide or “GC-B”) is a 32 amino acid polypeptide secreted by the ventricles of the heart in response to excessive stretching of myocytes (heart muscles cells) in the ventricles. At the time of release, a co-secreted 76 amino acid N-terminal fragment (NT-proBNP) is also released with BNP. Tests showing elevated levels of BNP or NT-pro-BNP in the blood are used as a diagnosis of heart failure and may be useful to establish prognosis in heart failure, as both markers are typically higher in patients with worse outcome. Both BNP and NT-pro-BNP have been approved as a marker for acute congestive heart failure (CHF). The plasma concentrations of BNP and its precursor NT-pro-BNP are increased in patients with asymptomatic and symptomatic left ventricular dysfunction.

(Serum) Creatinine

Creatinine and its use as bioamarker is known in the art.

Creatinine is created from creatine, a compound found almost exclusively in muscle, at a relatively constant rate. It leaves the muscle and enters the blood, where it is subsequently removed by the kidneys. Most of the creatinine enters the urine after being filtered by the glomeruli (some is secreted) and the remaining amount accumulates in the serum or plasma. If the kidneys lose their ability to filter blood (GFR decreases), more creatinine will accumulate and serum or plasma creatinine will rise. As a result, creainine is an indirect marker of glomerular filtration rate (GFR) or the functional capacity of the kidneys. Furthermore, creatinine levels may increase when ACE inhibitors (ACEI) or angiotensin-II receptor blockers (ARBs) are used in the treatment of chronic heart failure (CHF).

Preferably the assessment of at least one further biomarker for heart failure comprises measuring the concentration of at least one further biomarker in a biological sample of the subject.

Preferably the concentration of BNP, NT-pro-BNP, (serum) creatinine, sCD40L, PAPP-A, TnT, MPO, VEGF and/or P1GF is measured.

In a preferred embodiment the sample for assessing the further biomarker is the same biological sample than the biological sample for determining the OPN concentration, preferably the same plasma sample.

The method preferably further comprises comparing the measured concentration of the at least one further biomarker with a reference value.

The reference value is the biomarker concentration of a control sample or a biomarker cut-off value.

Preferably, the biomarker cut-off value was determined by a statistical classification method, preferably receiver operating curve (ROC) analysis, from biological samples of a patient group, preferably the same patient group that was used for determining the OPN cut-off value.

In a preferred embodiment the reference value is a NT-pro-BNP cut-off value.

A preferred NT-pro-BNP cut-off value is higher than 1.500 ng/l plasma, preferably higher than 1.800 ng/l plasma, more preferably the NT-pro-BNP cut-off value is 2093 ng/l plasma.

The preferred NT-pro-BNP cut-off value is 2093 ng/l plasma and was determined by ROC analysis. The same patient group was used as for determining the OPN cut-off value.

In a preferred embodiment the OPN concentration is compared with an OPN cut-off value. And the at least one further biomarker is NT-pro-BNP. The NT-pro-BNP concentration is also compared with an NT-pro-BNP cut-off value.

In the above patient or study group:

With both biomarkers below the cut-off value survival rates after 12, 24, 36 and 48 months were 97%, 94%, 91% and 88%. If only osteopontin values were above the cut-off value in a combined analysis survival rates were 90%, 81%, 71% and 60%. When only NT-pro-BNP was measured above the cut-off value, while osteopontin values were below the cut-off value of 929 ng/ml, survival rates at 12, 24, 36 and 48 months were 80%, 65%, 58% and 49%. However, as soon as both biomarkers exceed the cut-off values survival rates were only 57%, 41%, 30% and 27% at the respective time points as mentioned above. See also Table 2.

This corresponded to estimated 4-year mortality rates in patients with both biomarkers below the cut-off value of 12% compared to 73%, when both markers were elevated above the cut-off value (HR 98; 95%-CI 39-246; p<0.0001). In the lower NT-pro-BNP group, an osteopontin level above 929 ng/ml raised the estimated 4-year mortality to 40% compared to 12% in patients with a low osteopontin (HR 6.5; 95%-CI 2.4-17.4; p<0.001). Similarly, high osteopontin values in patients with NT-pro-BNP levels above the cut-off, increased the estimated 4 year mortality rate from 49% to 73% (HR 2.2; 95%-CI 1.2-3.9; p<0.01).

See also the Examples and FIG. 5.

TABLE 2

| | OPN conc. | | | |
|---|---|---|---|---|
| | <cut-off value | >cut-off value | <cut-off value | >cut-off value |
| Estimated survival | NT-pro-BNP conc. | | | |
| rates [%] | <cut-off value | <cut-off value | >cut-off value | >cut-off value |
| After 12 months | 97 | 90 | 80 | 57 |
| After 24 months | 94 | 81 | 65 | 41 |
| After 36 months | 91 | 71 | 58 | 30 |
| After 48 months | 88 | 60 | 49 | 27 |
| estimated 4-year | 12 | 40 | 51 | 73 |
| mortality rates [%] | | | | |

Thus, a OPN concentration below the cut-off value of 929 ng/ml plasma and a NT-pro-BNP concentration below the cut-off value of 2.093 ng/1 plasma is predictive of a survival of:
more than 90%, more preferably about 95% of patients after 12 months;
more than 90% of patients after 24 months;
about 90% of patients after 36 months; and
more than 80%, more preferably about 85% of patients after 48 months.

Thus, a OPN concentration above the cut-off value of 929 ng/ml plasma and a NT-pro-BNP concentration below the cut-off value of 2.093 ng/1 plasma is predictive of a survival of:
about 90% of patients after 12 months;
about 80% of patients after 24 months;
about 70% of patients after 36 months; and
about 60% of patients after 48 months.

Thus, a OPN concentration below the cut-off value of 929 ng/ml plasma and a NT-pro-BNP concentration above the cut-off value of 2.093 ng/1 plasma is predictive of a survival of:
about 80% of patients after 12 months;
more than 60%, more preferably about 65% of patients after 24 months;
more than 50%, more preferably about 55% of patients after 36 months; and
more than 40%, more preferably about 45% of patients after 48 months.

Importantly, a OPN concentration above the cut-off value of 929 ng/ml plasma and a NT-pro-BNP concentration above the cut-off value of 2.093 ng/1 plasma is predictive of a survival of:
more than 50%, more preferably about 55% of patients after 12 months;
about 40% of patients after 24 months;
about 30% patients after 36 months; and
more than 20%, more preferably about 25% of patients after 48 months.

### Methods for Determining the Concentration of OPN and Further Biomarkers

Methods for determining the concentration of a protein and/or other compounds in biological samples are known in the art and can be used for determining the concentration of OPN and further biomarkers in the biological samples, preferably plasma samples.

A specifically chosen procedure has to be as sensitive as the detection limit of OPN and optional the detection limit of a further biomarker will require.

The concentration of OPN and the at least one further biomarker is preferably carried out by using an immunological method or an immunocytological method and molecules binding to OPN and the biomarker.

Preferred immunological methods are ELISA, sandwich enzyme immunoassays and solid phase-based immunoassays.

Preferred molecules that specifically bind to OPN or the biomarkers are antibodies, monoclonal antibodies, polyclonal antibodies, and their fragments, such as Fab, Fv, scFv, diabodies.

Preferably the molecules that specifically bind to OPN or the biomarkers carry detectable labels. Suitable labels are known in the art and comprise radioactive labels, such as radio-isotopes, chromogenic dyes, fluorescent dyes, enzymes, cofactors, enzyme substrates and gold beads.

The molecules that specifically bind to OPN or the biomarkers can furthermore be coupled to solid phases and matrices. Preferred solid phases and matrices are resins, column materials, ELISA plates, magnetic particles and beads, gold beads.

Thus, a preferred method for providing a diagnosis, prognosis and/or risk stratification of a subject with heart failure according to the present invention, preferably comprises the following steps:
a) obtaining a biological sample from the subject;
b) determining the concentration of osteopontin (OPN) in said sample,
c) comparing the measured osteopontin concentration with at least one reference value, and
d) optional, assessing at least one further biomarker for heart failure.

### Uses and Kits According to the Invention

The present invention provides the use of the methods according to the present invention for identifying of patients or patient subgroups with elevated OPN concentrations, such as osteopontin plasma or serum concentrations, which suffer from a significantly higher cardiac risk.

This use provides risk stratification for these patients and patient subgroups.

The present invention provides the use of osteopontin as marker for diagnosis, prognosis and/or risk stratification of a subject with heart failure.

The present invention furthermore provides the use of the determination of the osteopontin plasma concentration in a biological sample of a subject for diagnosis, prognosis and/or risk stratification of heart failure.

In a preferred embodiment osteopontin or the determination of the osteopontin plasma concentration is used together with at least one further biomarker for heart failure, preferably NYHA stage, 6 minute walk test, maximum oxygen intake assessed during ergospirometry (VO₂ max), age, brain natiuretic peptide (BNP), NT-pro-BNP, soluble CD40 ligand (sCD40L), PAPP-A, troponin T (TnT), MPO, VEGF and/or P1GF and creatinine, in particular serum creatinine.

The present invention furthermore provides a kit for performing the methods and the uses according to the present invention, wherein the kit comprises elements enabling for specifically quantifying:
the OPN concentration, preferably the OPN plasma concentration, in a biological sample of a subject, and
optionally, the concentration of at least one further biomarker, preferably brain natiuretic peptide (BNP) or NT-pro-BNP, in said biological sample.

The elements especially enable to distinguish between patients and patient subgroups below or above OPN reference values, such as the OPN cut-off value.

Preferably, the kit comprises at least one antibody specific for osteopontin that is suitable for an ELISA assay and/or an osteopontin standard.

Furthermore, the kit preferably comprises instructions for interpreting the results of the OPN concentration and optional the at least one further biomarker concentration with respect to providing a diagnosis, prognosis and/or risk stratification of the subject whose biological sample was analyzed, such as for identifying patients or patient subgroups with elevated OPN concentrations, which suffer from a significantly higher cardiac risk.

The kit can furthermore comprise further components and/or suitable excipients.

The terms, such as “heart failure” and “diagnosis” and “prognosis” and “risk stratification”, are as defined above.

In heart failure, prognostic evaluation is critical in order to identify patients at highest risk for subsequent decompensation and death. Despite the availability of clinical criteria and the biomarker BNP, there is still considerable uncertainty in the prediction of prognosis. Consequently, there is great interest in new biomarkers that improve risk stratification.

Osteopontin, a glycoprotein that can be detected in plasma, was found upregulated in several animal models of cardiac failure, and may thus represent an attractive candidate molecule.

Osteopontin is associated with the severity of heart failure and indicates an adverse prognosis. Therefore, osteopontin plasma levels were analyzed in a large series of patients with chronic heart failure due to dilated or ischemic cardiomyopathy.

Here, we report that osteopontin plasma levels are indeed significantly elevated in patients with chronic heart failure. Moreover, OPN levels correlate with disease severity and independently predict mortality, suggesting that osteopontin is a useful novel biomarker for risk stratification of patients with heart failure.

OPN is a novel biomarker for risk stratification of patients with heart failure, because:
Osteopontin levels are significantly increased in patients with heart failure-irrespective of the underlying etiology;
Osteopontin levels correlate with the severity of heart failure;
Osteopontin predicts mortality in patients with heart failure;
Osteopontin has an additive value of in predicting mortality of heart failure patients.

Our data show that plasma levels of osteopontin are associated with heart failure severity as well as mortality, rendering this novel biomarker useful in the risk stratification of heart failure patients.

In this regard, recent reports have defined benchmarks for the assessment of novel cardiovascular biomarkers (14, 15): The clinical potential of a new marker can be evaluated by three fundamental questions: (1) Can the clinician measure it? (2) Does it add new information? (3) Does it help the clinician to manage patients?

First, plasma levels of osteopontin can easily and reproducibly be measured by the use of commercially available ELISA kits. Secondly, our results show that osteopontin as an independent predictor of 4 year mortality adds significant information in the risk assessment of patients with heart failure. We have also provided evidence that in patients with a given NT-pro-BNP, osteopontin levels markedly alter the prediction of 4-year mortality. The risk of death within 48 months quintuples in patients assigned to a low-risk group according to their NT-pro-BNP levels, when osteopontin is measured above its ROC-defined cut-off value. Even in the setting of already significantly elevated NT-pro-BNP, a high osteopontin level still confers an additional increase in the 4-year mortality risk, reaching 73% when both markers are combined. Taken together, it appears that osteopontin provides complementary prognostic information beyond traditional markers and thus improves risk stratification in patients with heart failure.

This invention demonstrates that osteopontin plasma levels are significantly elevated in patients with systolic heart failure. Moreover, osteopontin levels also provide prognostic information independent of established clinical and biochemical markers, such as NYHA stage and NT-pro-BNP.

Risk stratification with clinical parameters such as symptom severity and left ventricular ejection fraction is critical to identify those patients with heart failure at greatest risk for subsequent decompensation and death. Recently, biomarkers such as BNP have been shown to add clinically useful information in the management of heart failure patients.²⁴ However, even when clinical findings are combined with BNP levels, there is still considerable variation in the outcome. Thus, it is highly unlikely that a single marker will provide all the information needed for clinical decision making, and an integrated “multimarker strategy” may be preferable. As a consequence, there is still great interest in new biomarkers that complement existing diagnostic tools and may facilitate risk stratification in patients with heart failure. In this regard, we show that increased plasma levels of osteopontin are significantly associated with heard failure severity. Although patients with significantly impaired systolic function and NYHA class I or II symptoms revealed only mild increases in osteopontin levels, patients in NYHA class III or IV revealed a marked induction, which suggests that osteopontin is a marker for advanced heart failure. Moreover, osteopontin emerged as an independent predictor of 4-year death and added significant information for the risk assessment of patients with heart failure. We also provide evidence that even in patients with a given NT-pro-BNP level, osteopontin levels markedly altered the prediction of 4-year death. The risk of death within 48 months was almost 6-fold greater in patients assigned to a low-risk group according to their NT-pro-BNP levels and whose osteopontin levels were above the cutoff value. Even in the setting of an already significantly elevated NT-pro-BNP, a high osteopontin level still conferred an additional increase in the 4-year death risk, which reached 73% when both markers were combined. In contrast, the death rate was only 12% when both markers were measured below their respective cutoff values. Taken together, osteopontin provides complementary prognostic information beyond that of traditional markers and thus improves risk stratification in patients with heart failure.

In summary, the present invention shows for the first time that osteopontin plasma levels are not only elevated in heart failure patients with left ventricular dysfunction but also correlate with disease severity and the risk for subsequent death. The present data demonstrate that osteopontin expands the prognostic power of established biomarkers in heart failure, such as NT-pro-BNP.

### Further Uses of OPN

Osteopontin can be used for the diagnosis, prognosis and/or risk stratification of further cardiovascular entities, preferably patients with pulmonary arterial hypertension and the cardiac rhythm disorders as well as cardiac amyloidosis.

As mentioned above basic and clinical research provided evidence that myocardial osteopontin expression is increased in the setting of increased left ventricular work load (7-9). As of yet there is not much information available if osteopontin is also involved in right ventricular remodelling. Plasma levels of osteopontin can be elevated and contain prognostic information in patients with primary pulmonary hypertension (PAH), and also predict favourable responses towards medical interventions.

For details, see Example 3, which shows for the first time that plasma levels of OPN are elevated in patients with chronic pulmonary arterial hypertension. Therefore, the novel biomarker OPN can improve the non invasive monitoring of right ventricular dysfunction and remodelling in patients with PAH. (see also FIGS. 9 to 13).

Since local osteopontin expression generally correlates with the amount of organ fibrosis (11), plasma levels of osteopontin are an indicator for the occurrence of cardiac rhythm disorders. Osteopontin plasma levels can correlate with the frequency and executed therapies in ICD (internal cardioverter defibrillator) carrier.

The inventors furthermore found that osteopontin can show cardiac involvement in case of systemic amyloidosis. In this disease, primarily (multiple myeloma) or secondarily (chronic inflammation, malignant tumor), there occurs an excess production of a protein (amyloid) that cannot be further degraded and that can be characterized by its specific staining characteristics (e.g. Congo Red staining). A possible consequence is deposition of amyloid in the interstice of the heart accompanied by formation of a distinct thickening of the heart walls. The clinically appearance is a rapidly progressive restrictive cardiomyopathy. So far, the “gold standard” for diagnosis is endomyocardial biopsy (which is an invasive method). As a non-invasive method, there exists the possibility of using trans-thoracic echocardiography for indicating cardiac involvement in case of systemic amyloidosis. Thickening of the heart walls is also accompanied by a distinct interstitiell fibrosis. The inventors are, thus, of the opinion that osteopontin is of diagnostic and prognostic value in case of cardiac amyloidosis. Samples of about 100 patients with cardiac amyloidosis as well as systemic amyloidosis without cardiac involvement were measured.

The following example illustrate the present invention without, however, limiting the same thereto.

### BRIEF DESCRIPTION OF THE DRAWINGS

FIG. 1. Molecular structure of osteopontin (OPN)

OPN contains an RGD (arginine-glycin-aspartate) binding sequence that mediates interaction with several surface receptors, e.g. integrins, including β1-integrin, as well as a thrombin cleavage site and a calcium binding site.

FIG. 2. Induction of myocardial OPN mRNA in animal models of myocardial hypertrophy.

Hearts of mice with a cardiac-specific transgenic expression of calcineurin A or chronic stimulation with angiotensin II via an osmotic mini pump were removed and analyzed for myocardial expression of osteopontin by quantitative PCR. In both animal models for cardiac hypertrophy and failure, osteopontin is markedly upregulated in wild type or sham operated animals, respectively.

FIG. 3. Study Design.

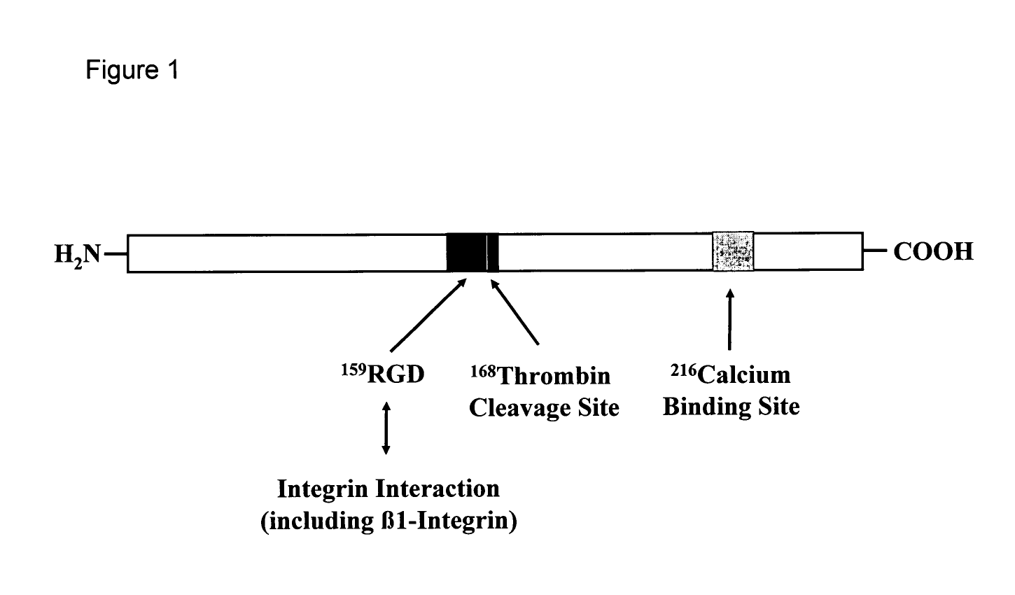

Flow chart illustrating the study design. According to the in- and exclusion criteria a database inquiry was performed at our heart failure clinic. 828 eligible plasma samples were identified which could be allocated to 420 individual patients. Osteopontin plasma levels were determined in all 420 patients by ELISA. In a subgroup of 223 patients NT-pro-BNP plasma samples were conducted from the same plasma sample and complete information of a 48 months follow up available. These patients were subsequently used for analysis of osteopontin's additive value in the risk stratification of heart failure patients.

FIG. 4. Estimated 4-year mortality rate according to osteopontin cut-off value.

Estimated 4-year mortality rates in patients with osteopontin levels above or below the cut-off value were 56.5% and 28.4%, respectively. Median survival of patients measured with osteopontin values above the cut-off was only 34 months whereas median survival in patients with osteopontin levels below the cut-off could not be calculated within 48 months of follow up.

FIG. 5. Additive value of osteopontin in 4-year mortality prediction.

Variation of osteopontin levels in a patient population with a NT-pro-BNP values below the cut-off defined by ROC analysis leads to a rise in estimated 4-year mortality rates from 12% to 39.4%. Similar results were obtained for the patient group with markedly elevated NT-pro-BNP values. Depending on the osteopontin level, mortality rates rose from 50.4% to 73% in these patients. Median Survival in the group of patients with values for both biomarkers above the cut-off level was only 18 months. Subjects with high NT-pro-BNP and low osteopontin revealed a median survival of 46 months. Calculations of median survival in the low risk groups was not possible within 48 months of follow up.

FIG. 6. Modified Box-whisker plot comparing the 10th, 25th, 50th, 75th, 90th percentile of OPN plasma levels in controls and CHF patients.

FIG. 7. Osteopontin plasma levels according to the NYHA classification.

Multigroup analysis by ANOVA revealed significant differences in NYHA III and IV patients compared to all other groups suggesting that osteopontin is a potential marker of chronic heart failure, in particular advanced heart failure.

FIG. 8. A receiver operating curve (ROC) analysis of osteopontin plasma levels for the prediction of 4-year mortality in patients with chronic heart failure.

FIG. 9. Osteopontin plasma levels in patients with pulmonary arterial hypertension. Osteopontin is elevated in patients with pulmonary arterial hypertension.

FIG. 10. Osteopontin plasma level in relation to WHO functional class. Osteopontin correlates with the clinical severity of the disease PAH.

FIG. 11. Osteopontin plasma level in relation to right ventricular remodelling. Osteopontin correlates with the right ventricular enddiastolic diameter.

FIG. 12. Osteopontin plasma level in relation to right ventricular function. Osteopontin is increased in patients with RV dysfunction.

FIG. 13. Osteopontin correlates with echo parameters of the RV function. Osteopontin correlates with echo-cardiographical parameters of the RV function.

### EXAMPLES

### Example 1

### Methods

### 1. Study Population

### a) Heart Failure Patients

Heart failure patients were recruited from the specialized heart failure clinic of a large university hospital that serves as a tertiary referral center in southern Germany. Eligible patients were ≧18 years of age and revealed significantly reduced left ventricular systolic function with an ejection fraction of less than 40%. Patients with dilated cardiomyopathy or ischemic heart failure were both included. Because Angiotensin II extensively stimulates osteopontin expression in the heart, all patients had to be taking an ACE-Inhibitor or Angiotensin II receptor blocker. All examinees enrolled in the present study had to have been taking stable medication one month before inclusion. Patients with malignant or inflammatory diseases, history of organ transplantation and significant acute/chronic renal failure (serum creatinine>2 mg/dl) were excluded.

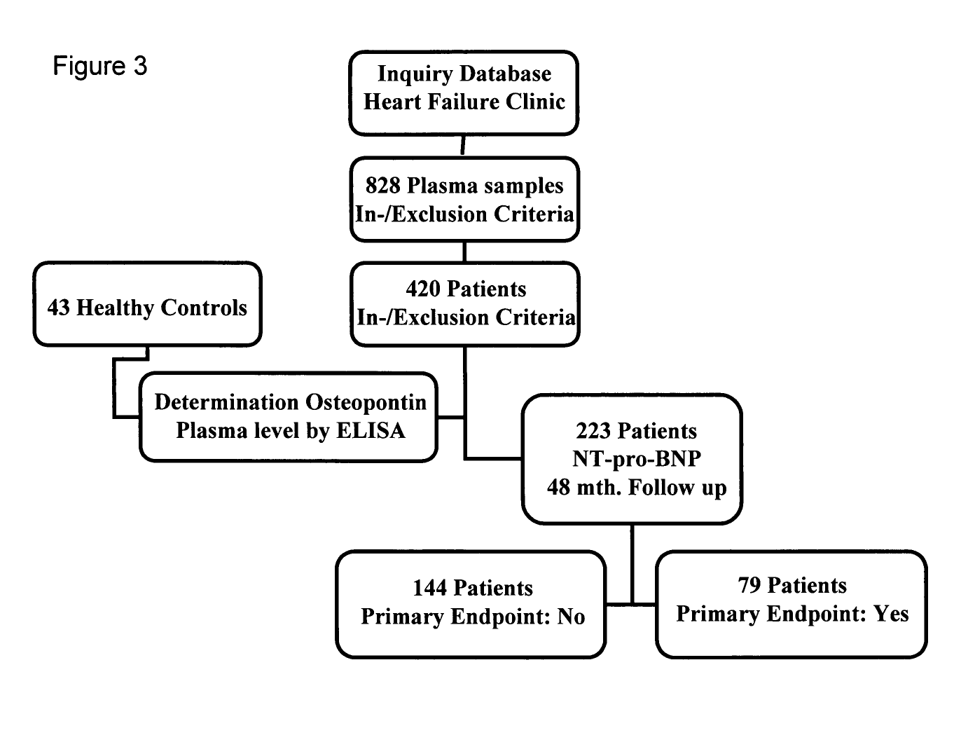

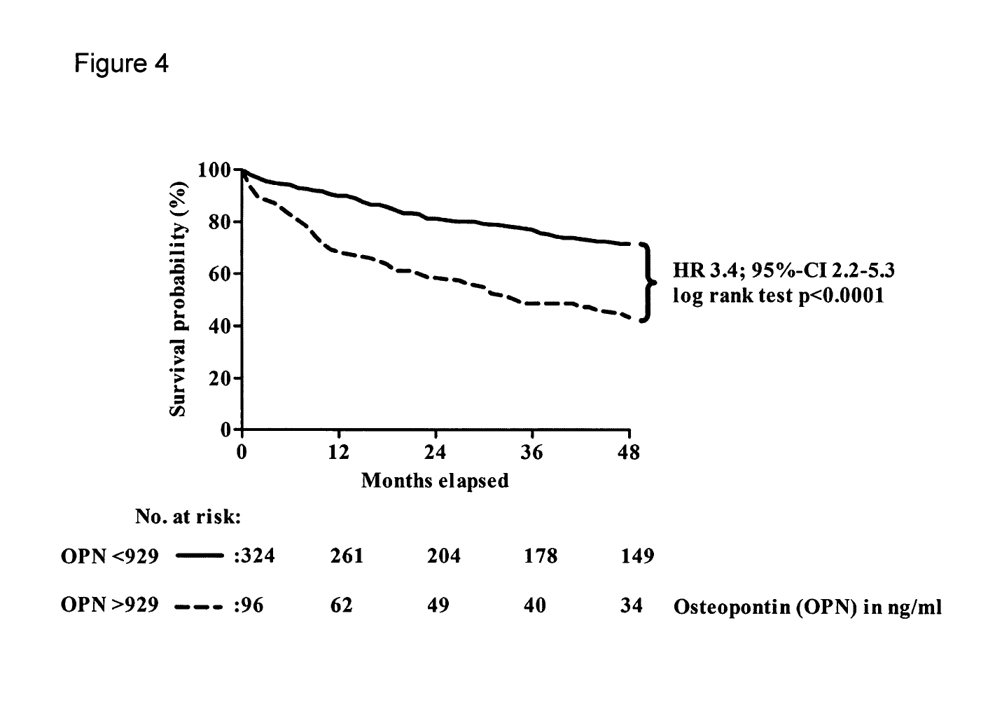

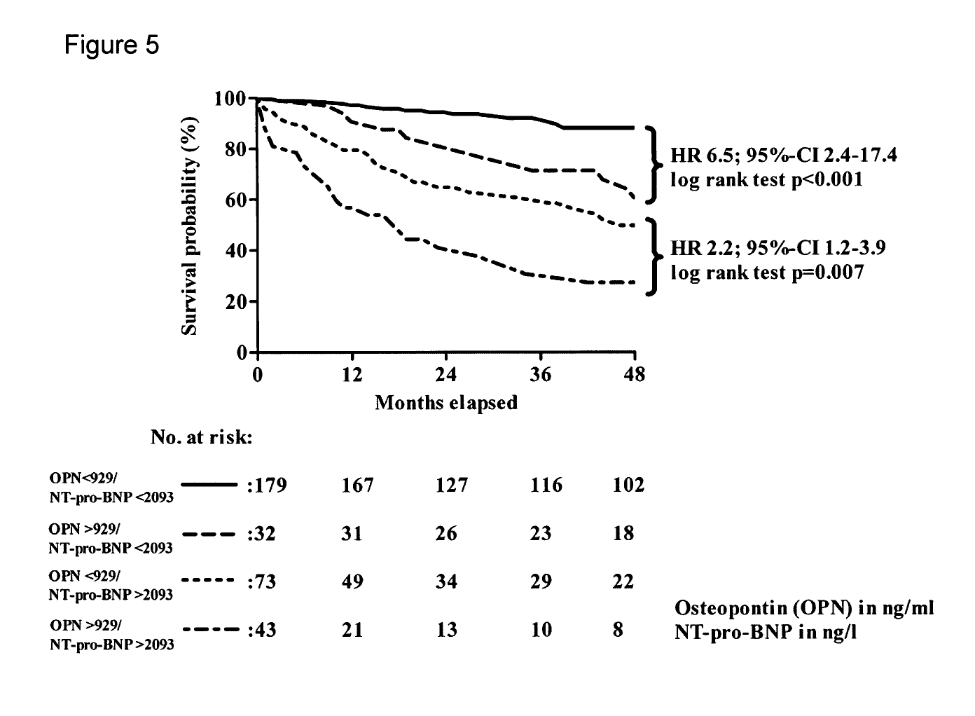

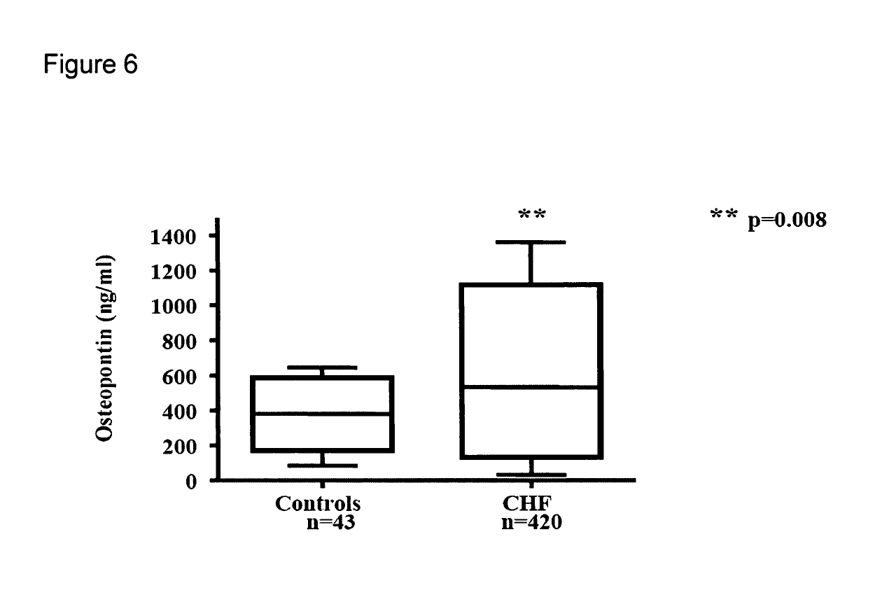

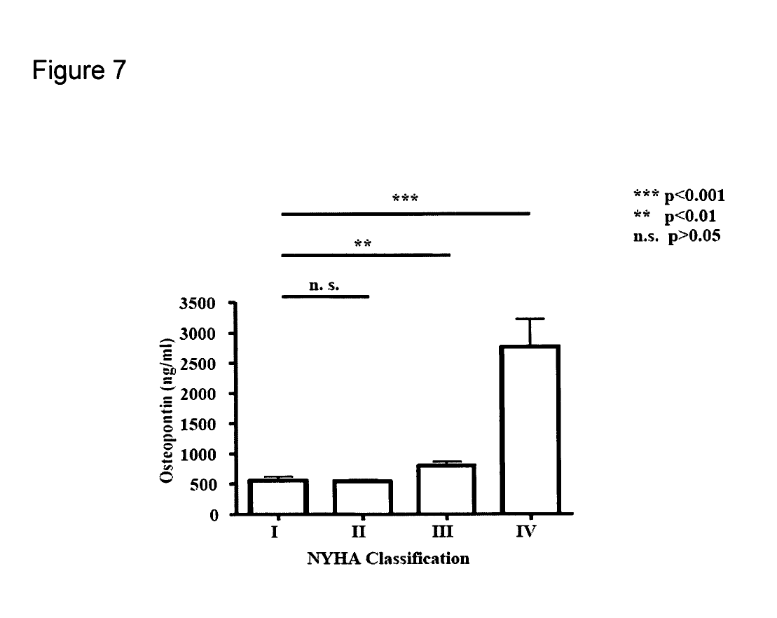

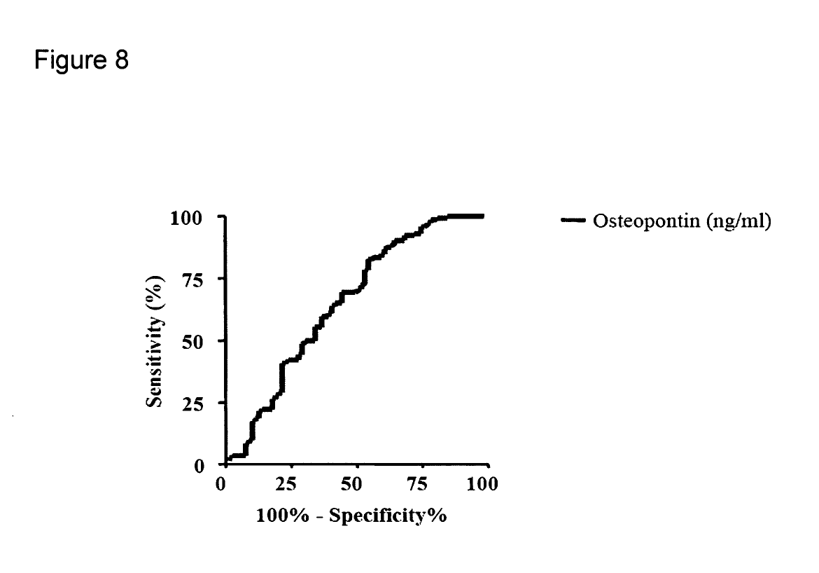

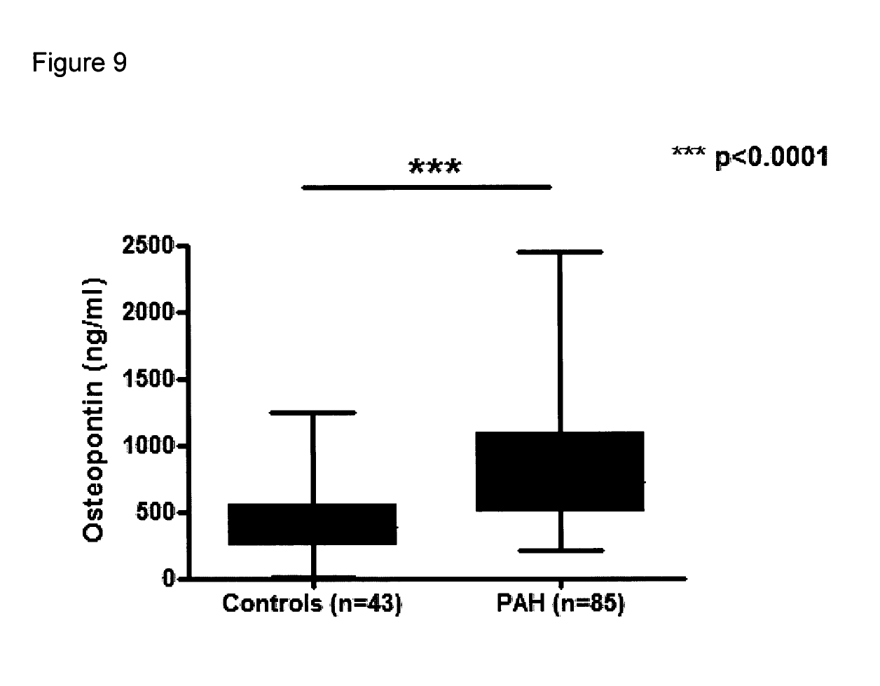

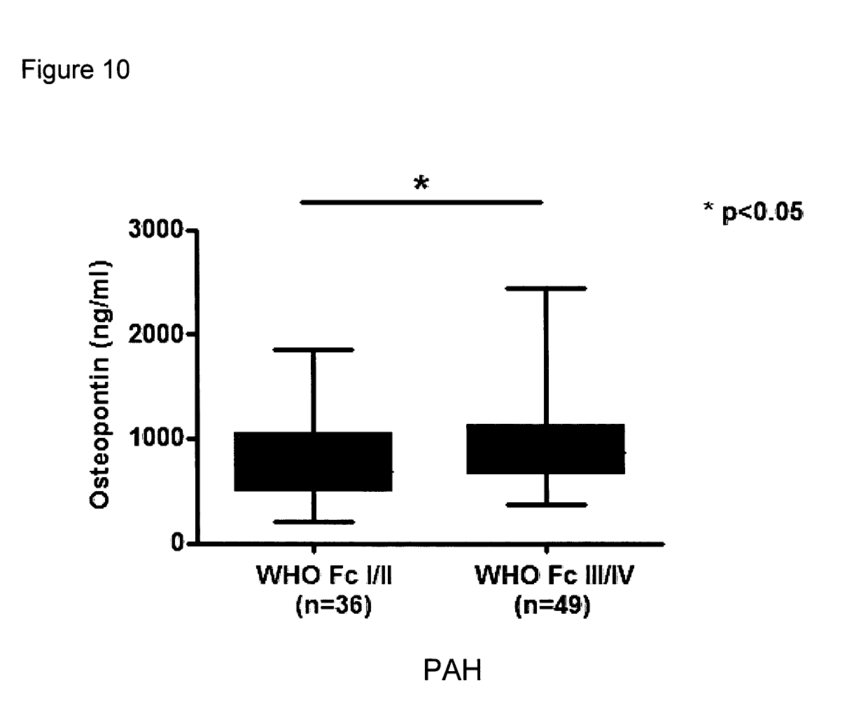

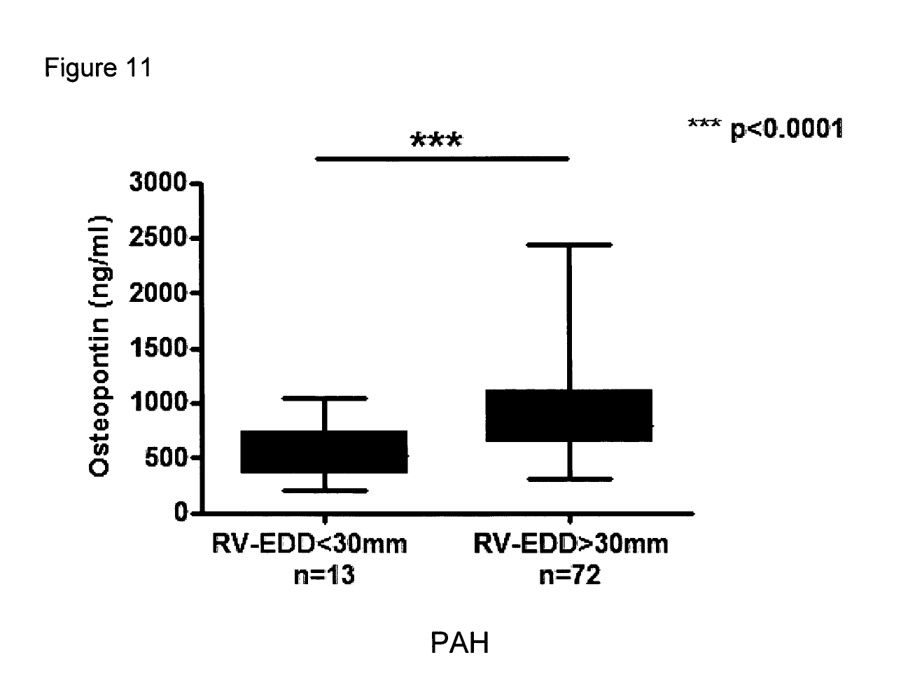

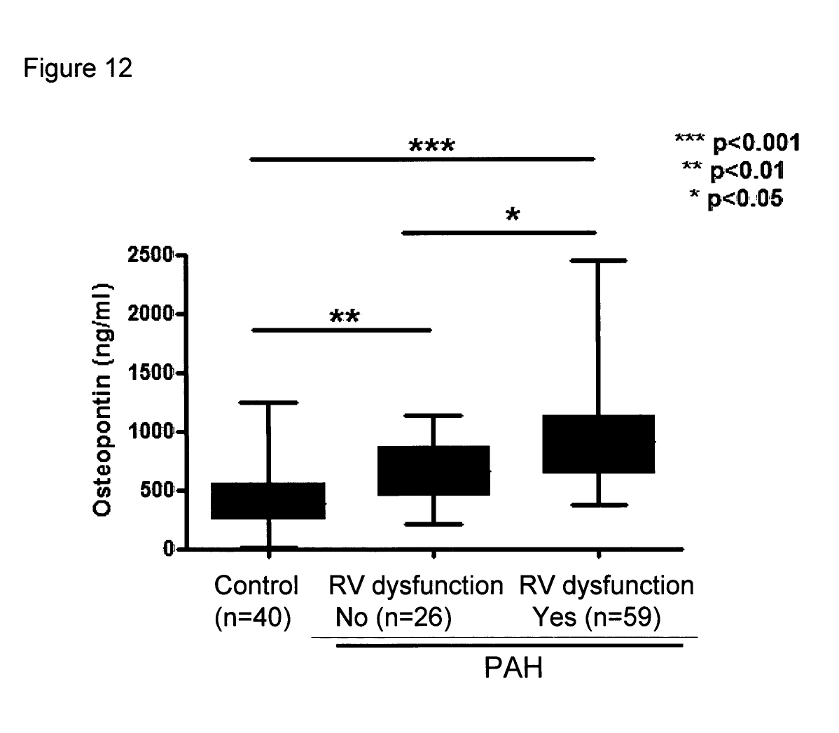

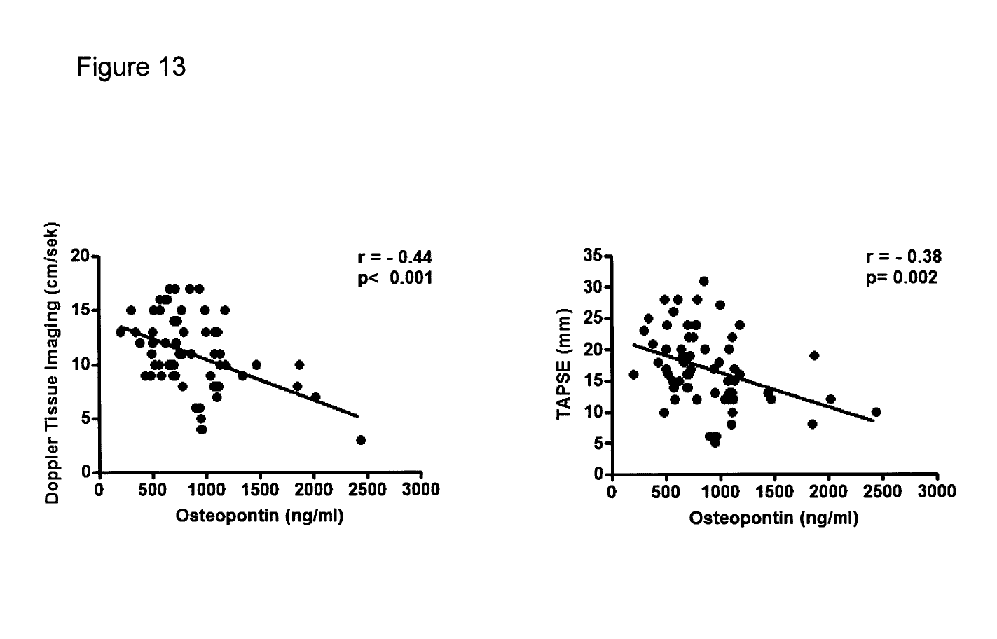

According to the in- and exclusion criteria we performed a database inquiry of our heart failure clinic biobank, which collects blood samples of all heart failure patients seen in the clinic. Plasma samples were considered for osteopontin testing, if they were drawn within the past 10 years (1996-2006). Thereby we obtained 828 eligible plasma samples that could be allocated to 420 individual patients. In patients with more than one plasma sample available, osteopontin was measured from the sample from which an NT-pro-BNP measurement had also been performed and/or which allowed complete analysis of 48 months' follow-up. Osteopontin plasma levels were determined by ELISA (see below) in all 420 patients at the most recent time point that allowed analysis of follow up information and at which a NT-pro-BNP measurement of the same plasma sample was conducted (available in 327 patients). To analyze a potential prognostic significance of osteopontin we defined all cause mortality within 48 months of follow up as the primary endpoint of our study. 4-year event rates in our heart failure patients were registered by yearly inquiry (outpatient visits and telephone calls) (see FIG. 3).

### b) Healthy Controls

EDTA plasma samples were also obtained from apparently healthy individuals. 43 subjects without signs of a significant heart disease were included and served as control group. Patients were recruited in our catheterization laboratory after undergoing coronary angiography for suspected coronary artery disease (CAD). Patients were only considered if invasive examination and echocardiography excluded CAD as well as systolic or diastolic dysfunction (defined as left ventricular enddiastolic pressures of less than 12 mmHg). Individuals who fulfilled these criteria were still excluded, if valvular heart disease or myocardial hypertrophy were evident on echocardiography. Further requirements for study enrollment was the absence of other acute or chronic diseases, as well as normal results on routine laboratory testing. All subjects included provided written informed consent and the study was approved by the local ethic committee of the University of Heidelberg.

### c) Study Participants

Our study group consisted of 420 heart failure patients and 43 healthy controls. In order to assay osteopontin plasma levels in patients with significant heart failure, we analysed 420 patients of whom 267 had dilated cardiomyopathy (64%) and 153 an ischemic origin of heart failure (36%). The median age of the heart failure group was 57 years and included 342 men (81%) and 78 women (19%). The control group was comprised of 17 men (39.5%) and 26 women (60.5%) with a median age of 59 years.

### 2. Biochemical Analyses

### a) Osteopontin Plasma Levels

Blood samples were drawn from control subjects and heart failure patients into a vacutainer coated with EDTA. Plasma samples were generated within 30 minutes of collection by centrifugation with 1.000 g for 10 minutes at 4° C. In order to avoid repetitive freeze and thaw cycles, different aliquots of one sample were generated, immediately frozen and stored at −80° C. until analysis, because osteopontin is sensitive to proteolytic degradation at higher temperatures. Plasma osteopontin levels were determined with a sandwich enzyme-linked immunosorbent assay (ELISA) using a commercially available kit (Immuno Biological Laboratories (IBL), Hamburg, Germany) according to the manufacturer's instructions. Human osteopontin is detected with this kit at a threshold of ≧5 ng/ml. Briefly, a 1:10 diluted test sample was incubated for 1 hour at 37° C. in wells precoated with an anti-human ostepontin antibody. After washing, 100 μl of horseradish peroxidase (HRP) conjugated anti-human osteopontin antibody was added to each well and incubated for 30 minutes at 4° C. After an additional washing step, tetramethyl benzidine (TMB) was used as a substrate and absorbance was measured at 450 nm with an automatic ELISA reader (Tecan Spectra, Crailsheim, Germany). Intra- and interassay coefficients of variation were less than 5% and 10%, respectively. Osteopontin measurements were carried out in duplicates by an investigator unaware of patients' characteristics and outcome.

### b) NT-pro-BNP Plasma Levels

NT-pro-BNP was measured from different aliquots of the same plasma sample. Measurements were carried out at the clinical core laboratory of the University Hospital Heidelberg using an ELISA assay (Roche Diagnostics, Mannheim, Germany).

### 3. Statistics

Data are presented as mean values (standard deviation), median (interquartile range) or as count and percentages. Continuous and categorical variables of heart failure patients and healthy control subjects were compared using the non-parametric Mann-Whitney U test or Fisher exact test, respectively. Linearity of categorical variables such as NYHA classification was confirmed and assessed by a method described by Hosmer and Lemeshow (16). The optimal plasma osteopontin and NT-pro-BNP cut-off value to predict an adverse outcome in our study population was calculated by a receiver-operating-characteristic (ROC) curve driven analysis. Estimates of the cumulative event rate were evaluated by the Kaplan-Meier method. Heart failure patients were compared according to the osteopontin cut-off value derived from the ROC analysis with the use of log-rank tests of the 4-year survival curves. Because the influence of other factors cannot be excluded by the univariate Kaplan-Maier analysis, univariate and multivariable analyses by Cox proportional hazards regression were performed as well, in order to identify independent predictors of 4-year mortality in our patient cohort. These models included all demographical, clinical and biochemical parameters of the study population. Only parameters with significant differences in univariate and multivariable testing are presented.

The inventors additionally calculated the univariate and multivariate Cox proportional hazards regression test with osteopontin either dichotomized according to median values or considered as a continuous variable (in order to further validate the cut-off value).

To test if osteopontin is of additive value in the risk stratification of patients with significantly impaired left ventricular function and known NT-pro-BNP levels, a subgroup of 327 (of a total of 420) patients was analyzed for whom both NT-pro-BNP and osteopontin measurements from the same plasma sample were available. Patients were categorized according to osteopontin and NT-pro-BNP cut-off values derived from the ROC analysis. Cumulative survival plots of the different groups were again calculated by the Kaplan-Meier method and compared with the use of the log-rank test.

Additionally the inventors performed the likelihood ratio test to confirm the additive value of osteopontin in the risk stratification of patients with chronic heart failure. The reduced model consisted only of NT-pro-BNP (as a continuous variable), whereas the full model included NT-pro-BNP and osteopontin (each as continuous variable).

A two-sided significance level of p<0.05 was specified for the comparison of heart failure patients and healthy controls, as well as for the comparison of the primary endpoint between heart failure patients classified according to demographical, clinical and biochemical parameters.

All statistical analysis were performed using Prism 5.0 (Graphpad Software, San Diego, USA) and MedCalc 9.3.0.0 (MedCalc, Mariakerke, Belgium) software.

### Example 2

### Results

### Clinical Characteristics of Patient Cohort and Control Subjects

Among 420 patients 267 had dilated cardiomyopathy (64%) and 153 an ischemic origin of heart failure (36%). The median age of the heart failure group was 57 years and included 342 men (81%) and 78 women (19%). The control group was comprised of 17 men (39.5%) and 26 women (60.5%) with a median age of 59 years.

The distribution of cardiovascular risk factors was similar in heart failure patients and controls (see Table 7 below).

Osteopontin Plasma Levels are Significantly Increased in Patients with Heart Failure-Irrespective of the Underlying Etiology

The median osteopontin plasma level in the control sample was 382 ng/ml (interquartile range: 257 ng/ml-540 ng/ml). Patients with systolic heart failure displayed a significantly higher (p=0.008) median osteopontin plasma level of 532 ng/ml (232 ng/ml-875 ng/ml). The upregulation of osteopontin plasma levels was independent of the underlying heart failure etiology. Patients with dilated cardiomyopathy revealed a median osteopontin plasma level of 577 ng/ml (151 ng/ml-954 ng/ml) that did not significantly differ from the median level of patients with ischemic cardiomyopathy (508 ng/ml (310 ng/ml-791 ng/ml)) (Table 3, see also FIG. 6). Neither in heart failure patients nor in controls a significant difference between male and female subjects was observed (data not shown).

TABLE 3
Plasma Osteopontin levels in patients with dilated or ischemic
cardiomyopathy in comparison to healthy control subjects.

| | Osteopontin ng/ml | |
|---|---|---|
| | Median | Interquartile range |
| Cardiomyopathy Total (n = 420) | 532 | 232-875 |
| Controls (n = 43) | 382 | 257-540 |
| p-Value | 0.008 | |
| Dilated Cardiomyopathy (n = 267) | 577 | 151-954 |
| Ischemic Cardiomyopathy (n = 153) | 508 | 310-791 |
| p-Value | 0.75 | |

3. Osteopontin Levels Correlate with the Severity of Heart Failure

Next, it was analyzed whether osteopontin plasma levels correlate with the severity of heart failure. Because plasma samples were obtained from patients treated in our outpatient clinic, the vast majority of the examinees had symptoms that placed them in NYHA functional class II (195; 46.5%) or III (161; 38.5%). Only a minority of the included patients were classified as NYHA I (56; 13%) or IV (8; 2%). Thus, patients with no or mild symptoms (NYHA I or II) were compared to patients with moderate to severe heart failure (NYHA III or IV).

Baseline characteristics of the 2 patient cohorts are illustrated in Table 7 (below) and revealed no significant differences with regard to demographic parameters, cardiovascular risk factors, current medication, or left ventricular function, however, patients with ischemic cardiomyopathy were more likely to have advanced heart failure.

Median osteopontin in patients with no or mild symptoms was 479 ng/ml (179 ng/ml-786 ng/ml) whereas patients with moderate to severe disease revealed a median osteopontin of 672 ng/ml (299 ng/ml-1.145 ng/ml; p<0.0001) (Table 4). Multigroup analyses by ANOVA revealed significant differences for both NYHA III and IV compared with all other groups (see FIG. 7). There was no significant difference between NYHA class I and II.

These findings suggest that osteopontin plasma levels are not only elevated in the presence of heart failure patients, but are also associated with disease severity, in particular with advanced heart failure.

TABLE 4
Osteopontin plasma levels according to NYHA Classification.

Osteopontin ng/ml

| NYHA Classification | Median | Interquartile range |
|---|---|---|
| I and II (n = 251) | 479 | 179-786 |
| III and IV (n = 169) | 672 | 299-1145 |
| P-Value | <0.0001 | |

4. Osteopontin Predicts Mortality in Patients with Heart Failure

Given that osteopontin levels were significantly elevated in patients with heart failure and also correlated with functional status, we sought to evaluate whether osteopontin could also provide prognostic information in our patient cohort. Therefore, a receiver operating curve (ROC) analysis was conducted to identify the optimal osteopontin plasma level for potential prediction of death within 48 months of follow up. The best cut-off value of osteopontin for the prediction of all cause mortality within 48 months in our study group was 929 ng/ml with a sensitivity of 46% and a specificity of 83% (see FIG. 8). The area under the curve (AUC) was 0.65 (95%-Confidence Interval (CI) 0.57-0.713; p<0.001). Subsequent survival analyses were performed according to this optimized threshold.

Baseline characteristics of the patients above or below the optimized osteopontin cut-off defined by ROC analysis are illustrated in Table 5. No significant differences were observed between patients with low and high OPN levels in respect to demographic factors, cardiovascular risk factors and medication, with the exception of a higher prevalence of dyslipidemia in the low OPN group. Moreover, neither the underlying etiology of heart failure (ischemic vs. dilated cardiomyopathy) nor the degree of left ventricular dysfunction was different in patients with low or high osteopontin levels, respectively. Yet, patients with NYHA class III and IV were significantly overrepresented in the high OPN group (NYHA III: 51.0% vs. 34.7%; p<0.01; NYHA IV: 7.0% vs. 0.3%; p<0.001), whereas functional NYHA class II was more prevalent in the group with OPN level below the cut-off value of 929 ng/ml (51.0% vs. 31.0%, p<0.001).

The mean follow up (±SE) of all 420 patients analyzed was 43±2 months. Mortality rates were calculated by Kaplan-Maier analysis according to the osteopontin cut-off value determined by the receiver operating curve (FIG. 4). Estimated 4-year mortality rates in heart failure patients with osteopontin levels above the cut-off value of 929 ng/ml were 56.5% vs. 28.4% in the population with lower osteopontin levels (Hazard Ratio 3.43; 95%-CI 2.2-5.3; p<0.0001). Median survival of patients measured with high osteopontin values was only 34 months, whereas median survival in patients with lower osteopontin levels was more than 48 months (FIG. 4). For 223 patients (53%) 48 months follow up was completed and information on OPN as well as NT-pro-BNP levels was available. 162 revealed OPN levels below 929 ng/ml, while 61 examinees had levels above the cut-off value. Death of any cause occurred in 43 patients with low OPN levels and 36 individuals with values above the cut-off, corresponding to significant differences in the 4-year event rate of 27% and 59% respectively (HR 2.9; 95%-CI 1.8-4.5; p<0.001).

Other factors significantly associated with 4-year mortality in our study included NT-pro-BNP, NYHA classification, creatinine and age (Table 6). Subsequent multivariable analysis by Cox proportional hazards regression dichotomized according to an optimal cut-off value derived from ROC analysis revealed that OPN independently predicts mortality (HR 2.3; 95%-CI 1.4-3.5; p<0.001). Besides OPN, NT-pro-BNP, serum creatinine and NYHA classification stage emerged as independent predictors in the multiple model (Table 6).

Similar results were obtained when the median osteopontin level (618 ng/ml) was used as reference value (see Table 8) or when osteopontin was included as a continuous variable in the multivariable Cox proportional hazards regression analysis (see Table 9).

TABLE 5
Patient characteristics according to Osteopontin cut-off value.

| | Osteopontin (ng/ml) | | |
|---|---|---|---|
| Parameter | <929 ng/ml | >929 ng/ml | P-Value |
| Demographics n (%) | | | |
| Patients | 324 | 96 | |
| Age | 55 ± 11 | 55 ± 11 | 0.28 |
| Female | 61 (19) | 17 (18) | 0.88 |
| Male | 263 (81) | 79 (82) | 0.88 |
| Risk Factor n (%) | | | |
| Hypertension | 150 (46) | 37 (38) | 0.20 |
| Dyslipidemia | 128 (39) | 25 (26) | 0.02 |
| Diabetes mellitus | 85 (26) | 31 (26) | 0.25 |
| Current/Former Smoker | 147 (45) | 38 (40) | 0.35 |
| Medication n (%) | | | |
| ACE-Inhibitor | 286 (88) | 87 (90) | 0.59 |
| AT-II Blocker | 38 (12) | 9 (10) | 0.59 |
| β-Blocker | 248 (77) | 68 (71) | 0.28 |
| Aldosteron-Antagonist | 147 (45) | 40 (42) | 0.56 |
| Statin | 133 (41) | 29 (30) | 0.06 |
| ASS | 27 (8) | 11 (11) | 0.41 |
| Phenprocoumon | 272 (84) | 79 (82) | 0.75 |
| Etiology n (%) | | | |
| Dilated Cardiomyopathy | 199 (61) | 68 (71) | 0.12 |
| Ischemic Cardiomyopathy | 125 (39) | 28 (29) | 0.12 |
| LV Ejection Fraction n (%) | | | |
| 40%-25% | 86 (26) | 18 (19) | 0.14 |
| <25% | 238 (74) | 78 (81) | 0.14 |
| NYHA Classification n (%) | | | |
| I | 46 (14) | 10 (11) | 0.39 |
| II | 165 (51) | 30 (31) | <0.001 |
| III | 112 (34.7) | 49 (51) | 0.004 |
| IV | 1 (0.3) | 7 (7) | <0.001 |

TABLE 6
Cox proportional-hazards regression for 4-year mortality rate in relation
to biochemical, demographical and clinical factors

| | Univariate Analysis | | Multivariable Analysis | |
|---|---|---|---|---|
| | HR (95%-CI) | p-Value | HR (95%-CI) | p-Value |
| OPN ± 929 ng/ml | 2.9 (1.8-4.5) | <0.0001 | 2.0 (1.3-3.2) | 0.003 |
| NT-pro-BNP ± | 5.1 (3.2-8.1) | <0.0001 | 3.1 (1.9-5.1) | <0.001 |
| 2093 ng/l | | | | |
| Creatinine ± 1.1 | 3.3 (2.1-5.1) | <0.0001 | 2.0 (1.2-3.2) | 0.005 |
| mg/dl | | | | |
| NYHA I-IV | 3.2 (2.2-4.7) | <0.0001 | 2.1 (1.4-3.0) | <0.001 |
| Age ± Median | 1.7 (1.1-2.7) | 0.02 | 1.4 (0.9-2.1) | 0.2 |

TABLE 8
Cox proportional-hazards regression for 4-year mortality rate in
relation to biochemical, demographical and clinical factors

| | Univariate Analysis | | Multivariable Analysis | |
|---|---|---|---|---|
| | HR (95%-CI) | p-Value | HR (95%-CI) | p-Value |
| OPN >/<618 | 2.0 (1.3-3.2) | 0.002 | 1.7 (1.03-2.6) | 0.03 |
| ng/ml* | | | | |
| NT-pro- | 3.5 (2.1-5.7) | <0.0001 | 2.3 (1.4-3.9) | 0.001 |
| BNP >/<1308 ng/l* | | | | |
| Creatinine >/<1.0 | 2.2 (1.4-3.5) | <0.0001 | 1.3 (0.8-2.1) | 0.25 |
| mg/dl* | | | | |
| NYHA I-IV | 3.2 (2.2-4.7) | <0.0001 | 2.5 (1.7-3.7) | <0.001 |
| Age >/<56 yrs* | 1.7 (1.1-2.7) | 0.02 | 1.4 (0.9-2.1) | 0.14 |

*Median values

TABLE 9
Cox proportional-hazards regression for 4-year mortality rate in relation to
biochemical, demographical and clinical factors

| | Univariate Analysis | | Multivariable Analysis | |
|---|---|---|---|---|
| | HR (95%-CI) | p-Value | HR (95%-CI) | p-Value |
| OPN ng/ml* | 1.0006 (1.0004-1.0007) | <0.0001 | 1.0003 (1.0001-1.0003) | 0.01 |
| NT-pro-BNP ng/l* | 1.0006 (1.0004-1.0007) | <0.0001 | 1.0003 (1.0001-1.0005) | 0.001 |
| Creatinine mg/dl* | 5.8629 (3.0315-11.339) | <0.0001 | 2.2699 (0.9623-5.3539) | 0.0625 |
| NYHA I-IV | 3.2155 (2.2088-4.681) | <0.0001 | 2.091 (1.4164-3.0861) | <0.001 |
| Age yrs* | 1.0306 (1.006-1.0555) | 0.01 | 1.0127 (0.9875-1.0385) | 0.33 |

*continuous variables

### 5. Additive Value of Osteopontin in Predicting Mortality of Heart Failure Patients

In order to test for a potential additive value of osteopontin in the prediction of mortality, patients were classified according to their NT-pro-BNP and osteopontin values. As mentioned above, we determined the optimal osteopontin and NT-pro-BNP plasma levels for the predicition of 4-year mortality by means of a ROC driven analysis.

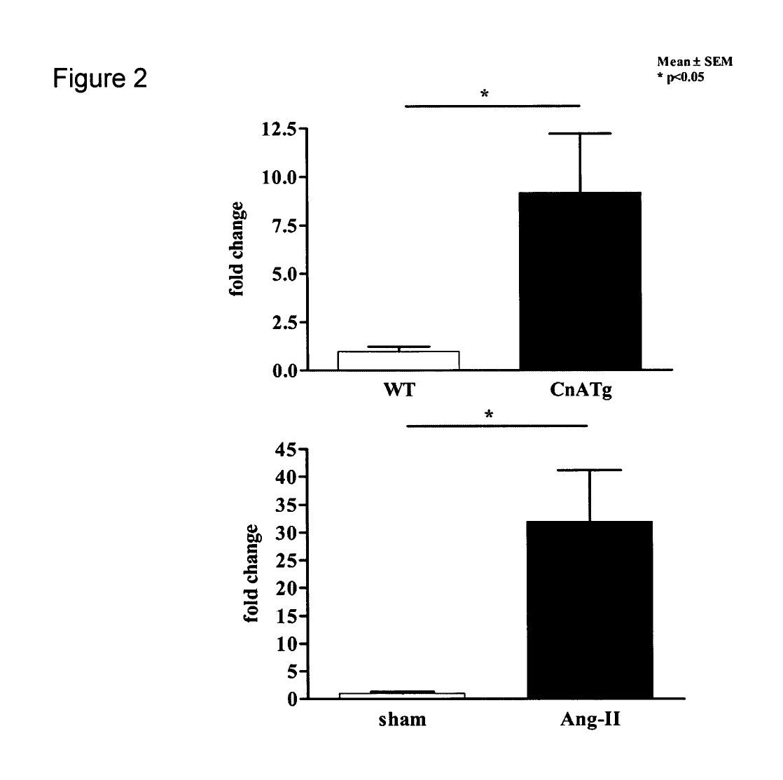

Mortality rates of the 327 patients for whom a simultaneous measurement of osteopontin and NT-pro-BNP was available, were calculated by the Kaplan-Meier method according to combined osteopontin and NT-pro-BNP cutoff values and compared using the log-rank-test. The estimated 4-year mortality rates in patients with both biomarkers below the cutoff was only 12% compared to 73%, when both markers were elevated above the cutoff (HR 98; 95%-CI 39-246; p<0.0001) (FIG. 2). In the lower BNP group, an osteopontin level above 929 ng/ml raised the estimated 4-year mortality to 39.4% compared to 12% in patients with a low osteopontin (HR 6.5; 95%-CI 2.4-17.4; p<0.001). Similarly, high osteopontin values in patients with BNP levels above the cutoff, increased the estimated 4 year mortality rate from 50.4% to 73% (HR 2.2; 95%-CI 1.2-3.9; p=0.007) (FIG. 2).

Because not all patients had the same follow-up range, we used a Cox proportional-hazards regression model to evaluate the prognostic information provided by osteopontin in chronic heart failure. A comparison of the reduced model with only NT-pro-BNP (as a continuous variable) and the full model with both NT-pro-BNP (as a continuous variable) and osteopontin (as a continuous variable) was made by the likelihood ratio test: For the reduced model (NT-pro-BNP), a likelihood ratio of G=17.94 was determined. P[χ²(1)>17.94]=0.00002 was calculated, which indicates that osteopontin provides additional prognostic information beyond that provided by NT-pro-BNP.

Thus, these results show that osteopontin carries additional and independent prognostic information in the risk stratification of patients with heart failure and impaired left ventricular function.

### Example 3

### Osteopontin Plasma Level Independently Predict Right Ventricular Dysfunction in Patients with Pulmonary Arterial Hypertension

The extracellular matrix protein Osteopontin (OPN) was found upregulated in several models of cardiac failure and appears to play an important role in myocardial remodeling. Moreover, the inventors showed herein that OPN plasma level are not only elevated in patients with left sided heart failure, but also correlated with an adverse prognosis. Since right ventricular dysfunction is an important predictor of morbidity and mortality in patients with pulmonary arterial hypertension (PAH), we now assessed the diagnostic power of OPN in this patient cohort.

We included 85 patients with PAH of different etiology in this study, while 43 healthy individuals of similar age and sex distribution served as controls. OPN plasma levels were determined by ELISA and assessed for correlation with clinical severity, echocardiographic parameters of right ventricular dysfunction and established biomarkers, including NT-pro-BNP.

OPN was significantly elevated in patients with pulmonary arterial hypertension compared to healthy controls (720 ng/ml vs. 382 ng/ml; p<0.0001). Furthermore, OPN levels were higher in patients with moderate to severe heart failure compared to patients with no or mild symptoms (WHO Fc III/IV 866 ng/ml vs. WHO Fc I/II 686 ng/ml; p=0.03). Patients with a right ventricular enddiastolic diameter above 30 mm displayed higher OPN plasma levels (789 ng/ml vs. 512 ng/ml; p<0.001). Additionally, plasma levels of OPN above the median (766 ng/ml) reliably predicted right ventricular dysfunction in our patient cohort (OR 6.0; 95%-CI 2.0-18.4; p=0.001). In a multivariate analysis including demographical, clinical and biochemical parameters such as NT-pro-BNP, OPN emerged also as an independent predictor of right ventricular dysfunction (OR 5.3; 95%-CI 1.1-27.1; p=0.04). See also FIGS. 9 to 13 and Table 10.

In summary, the data of the current study show for the first time that plasma levels of OPN are elevated in patients with chronic pulmonary arterial hypertension. Therefore, the novel biomarker OPN can also improve the non invasive monitoring of right ventricular dysfunction and remodelling in patients with PAH.

TABLE 10
Osteopontin is an independent predictor of RV dysfunction.

| | Simple Model | | | Multiple Model | | |
|---|---|---|---|---|---|---|
| Variable | OR | 95%-CI | p-Value | OR | 95%-CI | p-Value |
| Osteopontin* | 6.0 | 2.0-18.4 | 0.001 | 5.3 | 1.1-27.1 | 0.04 |
| NT-pro-BNP* | 7.1 | 2.1-24.7 | 0.002 | 7.0 | 1.1-49.0 | 0.04 |
| WHO Fc I-IV* | 8.6 | 2.6-28.2 | <0.001 | 6.4 | 1.1-40.8 | 0.04 |
| PAP Syst.* | 5.3 | 1.6-17.0 | 0.005 | 2.9 | 0.6-14.7 | 0.19 |
| 6 ‘Walk’ Test* | 0.41 | 0.1-1.3 | 0.12 | 0.8 | 0.1-4.2 | 0.79 |

*Median: Osteopontin ≧ 766 ng/ml; NT-pro-BNP ≧ 400 ng/l; PAP ≧ 62.5 mmHg; 6 MWT ≧ 456 m.

The features disclosed in the foregoing description, in the claims and/or in the accompanying drawings may, both separately and in any combination thereof, be material for realizing the invention in diverse forms thereof.

TABLE 7
Patient characteristics of the study collective and according to the NYHA classification

| | Study collective | | | NYHA-Classification | | |
|---|---|---|---|---|---|---|
| | Control | CHF-group | P-Value | NYHA I/II | NYHA III/IV | P-Value |
| Demographics n (%) | | | | | | |
| Patients | 43 | 420 | | 251 | 169 | |
| Age | 58 ± 12 | 55 ± 11 | 0.12 | 54 ± 11 | 57 ± 11 | 0.06 |
| Female | 26 (60.5) | 78 (19) | <0.001 | 46 (18) | 32 (19) | 0.9 |
| Male | 17 (39.5) | 342 (81) | <0.001 | 205 (82) | 137 (81) | 0.9 |
| Risk Factor n (%) | | | | | | |
| Hypertension | 22 (51) | 187 (45) | 0.42 | 115 (46) | 72 (43) | 0.55 |
| Dyslipidemia | 21 (49) | 153 (36) | 0.13 | 93 (37) | 60 (36) | 0.76 |
| Diabetes mellitus | 17 (39) | 116 (28) | 0.11 | 67 (27) | 49 (29) | 0.66 |
| Current/Former Smoker | 18 (42) | 185 (44) | 0.87 | 105 (42) | 80 (47) | 0.27 |
| Medication n (%) | | | | | | |
| ACE-Inhibitor | 15 (35) | 373 (89) | <0.001 | 220 (88) | 153 (91) | 0.43 |
| AT-II Blocker | 21 (49) | 47 (11) | 0.6 | 31 (12) | 16 (9) | 0.43 |
| β-Blocker | 20 (47) | 316 (75) | <0.001 | 194 (77) | 122 (72) | 0.25 |
| Aldosteron-Antagonist | 1 (2) | 187 (45) | <0.001 | 109 (43) | 78 (46) | 0.62 |
| Statin | 20 (47) | 161 (38) | 0.32 | 93 (37) | 69 (41) | 0.47 |
| ASS | 27 (63) | 38 (9) | <0.001 | 25 (10) | 13 (8) | 0.15 |
| Phenprocoumon | 1 (2) | 351 (84) | <0.001 | 207 (82) | 143 (85) | 0.59 |
| Etiology n (%) | | | | | | |
| Dilated Cardiomyopathy | — | 267 (64) | — | 177 (71) | 90 (53) | <0.001 |
| Ischemic Cardiomyopathy | — | 153 (36) | — | 74 (29) | 79 (47) | <0.001 |
| LV Ejection Fraction n (%) | | | | | | |
| 40%-25% | — | 104 (25) | — | 64 (25) | 40 (24) | 0.73 |
| <25% | — | 316 (75) | — | 187 (75) | 129 (76) | 0.73 |
| NYHA Classification n (%) | | | | | | |
| I | — | 56 (13) | — | 56 (22) | 0 | — |
| II | — | 195 (47) | — | 195 (78) | 0 | — |
| III | — | 161 (38) | — | 0 | 161 (95) | — |
| IV | — | 8 (2) | — | 0 | 8 (5) | — |

### REFERENCES

1. The American Heart Association. Heart Disease and Stroke Statistics-2005 Update. American Heart Association. Dallas, Tex.; 2005.
2. Haldeman G A, Croft J B, Giles W H, Rashidee A. Hospitalization of patients with heart failure: National Hospital Discharge Survey, 1985 to 1995. Am Heart J. 1999; 137: 352-360.
3. Bouvy M L, Heerdink E R, Leufkens H G, Hoes A W. Predicting mortality in patients with heart failure: a pragmatic approach. Heart. 2003; 89: 605-609.
4. Costello-Boerrigter L C, Burnett J C Jr. The prognostic value of N-terminal proB-type natriuretic peptide. Nat Clin Pract Cardiovasc Med. 2005; 2: 194-201.
5. Zugck C, Haunstetter A, Kruger C, Kell R, Schellberg D, Kubler W et al. Impact of beta-blocker treatment on the prognostic value of currently used risk predictors in congestive heart failure. J Am Coll Cardiol. 2002; 39: 1615-1622.
6. Denhardt D T, Noda M, O'Regan A W, Pavlin D, Berman J S. Osteopontin as a means to cope with environmental insults: regulation of inflammation, tissue remodelling and cell survival. J Clin Invest. 2001; 107: 1055-1061.
7. Okamoto H. Osteopontin and the cardiovascular system. Mol Cell Biochem. 2007, 300(1-2):1-7.
8. Singh K, Sirokman G, Communal C, Robinson K G, Conrad C H, Brooks W W, Bing O H L, Colucci W S. Myocaridal Osteopontin Expression Coincides With the Development of Heart Failure. Hypertension. 1999; 33: 663-670.
9. Graf K, Do Y S, Ashizawa N, Meehan W P, Giachelli C M, Marboe C C, Fleck E, Hsuch W A. Myocardial Osteopontin expression is associated with left ventricular hypertrophy. Circulation. 1997; 96: 3063-3071.
10. Stawowy P, Blaschke F, Pfautsch P, Goetze S, Lippek F, Wollert-Wulf B, Fleck E, Graf K. Increased myocardial expression of Osteopontin in patients with advanced heart failure. Eur JHeart Fail. 2002; 4: 139-146.
11. Satoh M, Nakamura M, Akatsu T, Shimoda Y, Segawa I, Hiramori K. Myocardial Osteopontin expression is associated with collagen fibrillogenesis in human dilated cardiomyopathy. Eur JHeart Fail. 2005; 7: 755-762.
12. Suezawa C, Kusachi S, Murakami T, Toeda K, Hirohata S, Nakamura K, Yamamoto K, Koten K, Miyoshi T, Shiratori Y. Time dependent changes in plasma osteopontin levels in patients with anterior-wall acute myocardial infarction after successful reperfusion: correlation with left-ventricular volume and function. J Lab Clin Med. 2005; 145: 33-40.
13. Young M F, Kerr J M, Termine J D, Wewer U M, Wang M G, McBride O W, Fisher L W. cDNA cloning, mRNA distribution and heterogeneity, chromosomal location, and RFLP analysis of human osteopontin (OPN). Genomics. 1990; 7(4):491-502.
14. Morrow DA, de Lemos J A. Benchmarks for the Assesment of Novel Cardiovascular biomarkers. Circulation. 2007; 115: 949-952.
15. Vasan R S. Biomarkers of Cardiovascular disease. Molecular Basis and Practical Considerations. Circulation. 2006; 113: 2335-2362.
16. Hosmer D W, Jr., Lemeshow S. Applied logistic regression. 2^{nd} ed. New York: Wiley & sons, 2000.

1. A method for providing a diagnosis, prognosis and/or risk stratification of a subject with heart failure, comprising:
a) providing a biological sample from the subject;
b) determining the concentration of osteopontin (OPN) in said sample,
c) comparing the determined OPN concentration with at least one reference value, and
d) optionally, assessing at least one further biomarker for heart failure.

2. The method according to claim 1, wherein the reference value is the OPN concentration of a control sample or an OPN cut-off value.

3. The method according to claim 1, wherein the OPN concentration is the OPN plasma concentration.

4. The method according to claim 2, wherein the control sample is selected from a biological sample of a control subject and an osteopontin standard.

5. The method according to any of claim 2, wherein the OPN concentration of a control sample is the median OPN concentration of control samples of a group of control subjects.

6. The method according to claim 2, wherein the OPN cut-off value was determined by a receiver operating curve (ROC) analysis from biological samples of a patient group.

7. The method according to claim 6, wherein the OPN cut-off value is higher than 850 ng/ml plasma.

8. The method according to claim 1, wherein the OPN concentration of the subject with heart failure is elevated compared to the reference value.

9. The method according to claim 1, wherein providing a diagnosis is determining heart failure.

10. The method according to claim 1, wherein providing a prognosis is selected from determining heart failure severity, risk for subsequent all-cause mortality and risk assessment of the subject with heart failure.

11. The method according to claim 10, wherein the risk for subsequent all-cause mortality is a 4-year mortality prediction.

12. The method according to claim 1, wherein heart failure is selected from the group consisting of chronic heart failure, systolic heart failure, dilated cardiomyopathy (DCM), ischemic cardiomyopathy, acute myocardial infarction, left ventricular dysfunction, and right ventricular dysfunction.

13. The method according to claim 1, further comprising the assessment of at least one further biomarker for heart failure, selected from the group consisting of NYHA stage, 6 minute walk test, maximum oxygen uptake assessed during ergospirometry (VO2 max), age, brain natriuretic peptide (BNP), NT-pro-BNP, creatinine, soluble CD40 ligand (sCD40L), PAPP-A, troponin T (TnT), MPO, VEGF and PIGF.

14. The method according to claim 13, wherein the assessment of at least one further biomarker for heart failure comprises measuring the concentration of at least one further biomarker selected from the group consisting of brain natriuretic peptide (BNP), NT-pro-BNP, creatinine, soluble CD40 ligand (sCD40L), PAPP-A, troponin T (TnT), MPO, VEGF and PIGF in a biological sample of said subject.

15. The method according to claim 14, further comprising comparing the measured concentration of the at least one further biomarker with a reference value.

16. The method according to claim 15, wherein the reference value is the biomarker concentration of a control sample or a biomarker cut-off value.

17. The method according to claim 16, wherein the reference value is a NT-pro-BNP cut-off value higher than 1.500 ng/l plasma.

18. The method according to claim 1, wherein the biological sample of the subject and/or the control sample is taken from a human.

19. The method according to claim 18, wherein the sample is selected from a bodily fluid, whole blood, plasma, serum, urine and cell culture suspensions or fractions thereof.

20. The method according to claim 19, wherein the sample is blood plasma or blood serum.

21. The method according to claim 19, wherein a coagulation inhibitor is added to peripheral blood.

22. The method according to claim 1, wherein determining the concentration of OPN and the at least one further biomarker is carried out by using an immunological method and molecules binding to OPN and the biomarker.

23. A method for identifying patients or patient subgroups with elevated OPN concentrations, which suffer from a significantly higher cardiac risk, wherein said method comprises the steps of claim 1.

24-29. (canceled)

30. A kit for performing a method according to claim 1, comprising elements for specifically quantifying:
the osteopontin concentration in a biological sample of a subject, and
optionally, the concentration of at least one further biomarker in said biological sample.

31. The kit according to claim 30, comprising at least one antibody specific for osteopontin that is suitable for an ELISA assay and/or an osteopontin standard.

32. A method for diagnosis, prognosis and/or risk stratification of further cardiovascular entities, wherein said method comprises the use of osteopontin.
